(19) United States
(12) Patent Application Publication
(10) Pub. No.: US 2024/0197981 A1
(43) Pub. Date: Jun. 20, 2024

(54) Wound Therapy System

(71) Applicant: 3M INNOVATIVE PROPERTIES COMPANY, St. Paul, MN (US)

(72) Inventors: Stephen M. Kennedy, St. Paul, MN (US); Gilles J.B. Benoit, Minneapolis, MN (US); Brian E. Brooks, St. Paul, MN (US); Kristine M. Kieswetter, San Antonio, TX (US); Susan L. Woulfe, Woodbury, MN (US)

(21) Appl. No.: 18/287,820

(22) PCT Filed: Apr. 25, 2022
(86) PCT No.: PCT/IB2022/053825

§ 371 Date: Oct. 20, 2023

Related U.S. Application Data
(60) Provisional application No. 63/201,319, filed on Apr. 23, 2021.

(51) Int. Cl. A61M 1/00; G16H 10/60

(52) U.S. Cl. CPC A61M 1/92; A61M 1/966; G16H 10/60; A61M 2205/3334; A61M 2205/3368

(57) ABSTRACT

An example system includes a memory and one or more processors in communication with the memory and configured to receive patient information and select at least one negative-pressure wound therapy (NPWT) parameter setting based on a causal model that determines 5 current causal relationships between a set of NPWT parameter settings and a set of effects of controlling the fluid at the wound site. The one or more processors are further configured to control the fluid at the wound site via the NPWT dressing based on the selected at least one NPWT parameter setting.

### TECHNICAL FIELD

The disclosure relates to wound therapy.

### BACKGROUND

Smart wound dressings, wound therapies, and wearable sensors are used to monitor healing and deliver therapeutic treatments to wounds. The therapeutic treatment may be adjusted based on monitoring of healing.

### SUMMARY

In general, the disclosure describes systems and techniques for selecting wound therapy parameter settings based on a causal model that determines current causal relationships between the wound therapy parameter settings and the effects of the wound therapy, e.g., delivered according to the wound therapy settings.

For example, a negative-pressure wound therapy (NPWT) system may be configured to receive patient information, including effects of controlling a fluid at a wound site, and select NPWT parameter settings for controlling the fluid at the wound site based on a causal model that determines current causal relationships between the NPWT parameter settings and the effects of controlling the fluid at the wound site, e.g., according to the selected NPWT parameter settings.

The systems and techniques disclosed may provide improved patient outcomes, e.g., via improved therapy control decisions. For example, the systems and techniques disclosed may implement sophisticated Deep Causal Learning (DCL) algorithms to carry out control decisions on the large and complex input data sets pertinent to the regulation of NPWT and wound irrigation/instillation therapy control parameters. A DCL-based feedback control system may enable multi-objective patient outcome improvement such as improving healing rate while reducing patient discomfort, wound infection, peri-wound maceration, and other undesirable complications.

In one example, this disclosure describes a method the includes receiving patient information; selecting at least one negative-pressure wound therapy (NPWT) parameter setting for controlling a fluid at a wound site via a NPWT dressing based on a causal model that determines current causal relationships between a set of NPWT parameter settings and a set of effects of controlling the fluid at the wound site; and controlling the fluid at the wound site via the NPWT dressing based on the selected at least one NPWT parameter setting.

In another example, this disclosure describes a system that includes a memory; and one or more processors in communication with the memory and configured to: receive patient information; select at least one negative-pressure wound therapy (NPWT) parameter setting based on a causal model that determines current causal relationships between a set of NPWT parameter settings and a set of effects of controlling the fluid at the wound site; and control the fluid at the wound site via the NPWT dressing based on the selected at least one NPWT parameter setting.

In another example, this disclosure describes a computer readable medium that includes instructions that when executed cause one or more processors to: receive patient information; select at least one negative-pressure wound therapy (NPWT) parameter setting based on a causal model that determines current causal relationships between a set of NPWT parameter settings and a set of effects of controlling the fluid at the wound site; and control the fluid at the wound site via the NPWT dressing based on the selected at least one first NPWT parameter setting.

In another example, this disclosure describes a system that includes a means for receiving patient info; a means for selecting at least one negative-pressure wound therapy (NPWT) parameter setting for controlling a fluid at a wound site via a NPWT dressing based on a causal model that determines current causal relationships between a set of NPWT parameter settings and a set of effects of controlling the fluid at the wound site; and a means for controlling the fluid at the wound site via the NPWT dressing based on the selected at least one NPWT parameter setting.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is an illustration depicting an example system, in accordance with the techniques described in this disclosure.

FIG. 2 is a block diagram illustrating an example computing device configured to execute a causal model, in accordance with the techniques of this disclosure.

FIG. 3 is a conceptual diagram illustrating a control system that selects therapy parameter settings via a causal model, in accordance with one or more techniques of this disclosure.

FIG. 4 is conceptual diagram illustrating example causal model, in accordance with one or more techniques of this disclosure.

FIG. 5 is a plot of example complex impedances of a tissue site at a predetermined frequency measured at a plurality of times after a wound occurs at a tissue site, in accordance with the techniques described in this disclosure.

FIG. 6 is a plot of example complex impedances of a tissue site at a predetermined frequency measured at a plurality of times after a wound occurs at a tissue site.

FIG. 7 is an illustration of an example scenario of wound measurements and NPWT parameter settings over a period of time, in accordance with the techniques described in this disclosure.

FIG. 8 is an illustration of another example scenario of wound measurements and NPWT parameter settings over a period of time, in accordance with the techniques described in this disclosure.

FIG. 9 is an illustration of another example scenario of wound measurements and NPWT parameter settings over a period of time, in accordance with the techniques described in this disclosure.

FIG. 10 is an illustration of another example scenario of wound measurements and NPWT parameter settings over a period of time, in accordance with the techniques described in this disclosure.

FIG. 11 is an illustration of another example scenario of wound measurements and NPWT parameter settings over a period of time, in accordance with the techniques described in this disclosure.

### DETAILED DESCRIPTION

In examples, the disclosure describes systems and techniques for selecting wound therapy parameter settings based on a causal model that determines current causal relationships between the wound therapy parameter settings and the effects of the wound therapy, e.g., delivered according to the wound therapy settings.

Sensor technologies may improve wound care and may enable and improve telemedicine-based healthcare paradigms. For example, negative pressure wound therapy (NPWT) and irrigation/instillation wound therapies system control parameters (e.g., pressure, flow and/or dwell time (in the case of instillation), irrigation/instillation solution temperature and content, etc.) may be regulated in order to improve patient outcomes (e.g., regulate control parameters to expedite healing and/or reduce patient discomfort based on feedback). In some examples, such regulation of control parameters may enable telemedicine by automating system regulation decisions (e.g., a clinician does not have to observe the patient and/or wound to decide to modify treatment parameters).

Some techniques may have an inability to process the complexity, dynamisms, and interplay of the data, and may therefore be limited in improving patient outcomes. For example, control decisions that regulate system control parameters in NPWT and irrigation/instillation therapies can be based on diverse, complex, real-time, and dynamic data sets (e.g., patient demographic information, patient medical history, on-demand user inputs, spatiotemporally dynamic sensor measurements of wound tissues, dynamic patient vital biometrics, etc.).

For example, a wound care specialist may periodically measure a geometry of the wound and determine whether healing has occurred and/or is occurring based on a reduction in wound area/volume over time. These periodic measurements require dressing removal and intimate inspection of the wound, e.g., inserting a ruler into the wound to assess its depth. Dressing removal may be disruptive to the wound bed and may add exposure time of the wound, e.g., to a non-sterile environment. If the wound care specialist determines that the wound is not reducing in area/volume, the wound specialist may alter the therapeutic strategy. For example, the wound care specialist may change NPWT to temporarily pause the NPWT, e.g., a “vac vacation.” In some examples, the wound care specialist may implement an instillation and/or irrigation regimen as part of the therapy, e.g., such that the wound is periodically filled with fluid for a set dwell time and drained via a new application of negative pressure (in the case of instillation) or continuously flushed with fluid concurrently with the application of negative pressure (in the case of irrigation) to clean out sluff and remove traces of microbials. In some examples, the wound care specialist may cause concentrated oxygen to be delivered topically to the wound bed in order to improve the oxygen gradient at the wound-tissue interface. As used herein, a “fluid” refers to any substance that deforms or “flows” when subjected to one or more external forces (e.g., pressure, gravity, etc.). Fluids, whether in a liquid state (e.g., saline solution, distilled water, etc.), a gaseous state (e.g., a mixture of gases such as atmospheric air, a gaseous element such as pure oxygen, etc.), a plasma state, or others can be used in accordance with various techniques described herein, such as NPWT.

In some examples, the wound care specialist may make a variety of control decisions that regulate system control parameters in NPWT and irrigation/instillation therapies based on experience, intuition, and intermittent subjective metrics. For example, the wound care specialist may determine, based on experience, intuition, and intermittent subjective metrics, how long to pause negative pressure, if and when to begin negative pressure after a pause and how long to apply the negative pressure, whether to cycle negative pressure pausing and the “on” and “off” times of the cycling, when to end negative pressure therapy, purge rate, whether to irrigate with fluid, whether to instill with fluid at predetermined “on” and “off” times of the cycling, the instillation fluid fill and/or concentration, instillation delivery rate, dwell time, and purge rate of each cycle, and any other suitable NPWT control decisions. In some examples, it may be difficult for a wound care specialist and/or current NPWT control schemes to determine causal relationships between control decisions and therapy outcomes and/or measures in order to base control decisions on objective criteria, e.g., based on cause and effect, rather than on subjective criteria, e.g., based on experience, intuition, and intermittent subjective metrics.

The systems and techniques disclosed may provide improved patient outcomes, e.g., via improved therapy control decisions. For example, the systems and techniques disclosed may implement sophisticated Deep Causal Learning (DCL) algorithms to carry out control decisions on the large and complex input data sets pertinent to the regulation of NPWT and wound irrigation/instillation therapy control parameters. A DCL-based feedback control system may enable multi-objective patient outcome improvement such as by improving healing rate while reducing one or more of patient discomfort, wound infection, peri-wound maceration, or other undesirable complications.

In some examples, a control system may monitor and adjust stimuli to improve and promote tissue health. For example, skin and/or wound health sensors may be combined with one or more wound treatments involving stimuli and control algorithms based on DCL to adjust the treatments to improve patient outcomes. The control system may receive algorithmic inputs, a DCL model and/or algorithm may be configured to select control parameters settings, e.g., NPWT parameters settings, and a wound therapy and/or treatment system, e.g., a NPWT system may be configured to provide a wound therapy based on the selected control parameters settings.

In some examples, the algorithmic inputs may include patient information. Patient information may include patient inputs, real-time and/or recorded patient biometric sensor data, real-time and/or recorded wound sensor data, and any other suitable patient and/or wound information. Patient inputs, e.g., user inputs received from a clinician and/or patient, may include patient demographics, patient health record data, input data related to patient discomfort, clinician inputs related to prognosis, lists of treatment options, and any other suitable patient input information. In some examples, patient input may enable clinician control over the treatment regimen. Real-time and/or recorded patient biometric sensor data may include blood pressure, heart rate, temperature, blood glucose levels, albumin, pre-albumin, tissue oxygen concentration, oxygenated hemoglobin levels, and the like. Real-time and/or recorded wound sensor data may include impedance-based wound monitoring, imaging, temperature and/or pressure measurements, and/or any other suitable wound sensor data.

In some examples, a DCL model and/or algorithm may be configured to process large, spatiotemporally dynamic, and often interrelated quantities of input data to regulate control parameters pertinent to treatment options. A DCL model and/or algorithm may be configured to determine current causal relationships between control parameters settings and the effects of providing a wound therapy based on the control parameters settings.

In some examples, a wound therapy and/or treatment system may include a NPWT and combined NPWT-instillation systems (e.g., V.A.C. VERAFLO™ Therapy) or a NPWT and combined NPWT irrigation systems. The term “negative pressure” refers to an absolute pressure that is lower than the absolute atmospheric pressure at the location of use of the device. A stated level of negative pressure in a region is therefore a relative measure between the absolute atmospheric pressure and the absolute pressure in the region. A statement that the negative pressure is decreasing means the pressure in the region is transitioning towards atmospheric pressure (e.g., the absolute pressure is increasing). Where numeric values are used a negative sign is placed in front of the numeric pressure value to indicate the value is a negative pressure relative to atmospheric pressure.

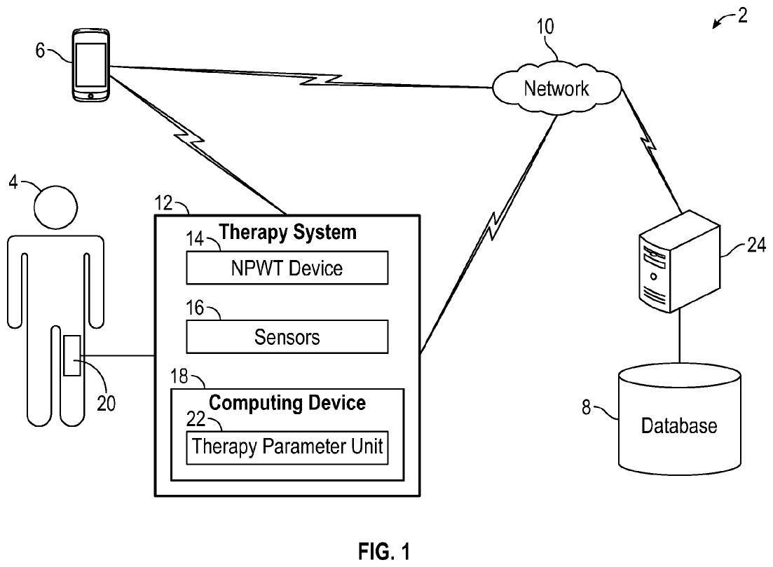

FIG. 1 is an illustration depicting an example system 2, in accordance with the techniques described in this disclosure. As illustrated in FIG. 1, system 2 includes patient 4, NPWT dressing 20, therapy system 12, and server 24 that may communicate via network 10.

Therapy system 12 may be configured to receive algorithmic inputs, select wound therapy control parameters settings based on a DCL model and/or algorithm, and deliver wound therapy via a wound therapy and/or treatment system, e.g., a NPWT system, according to the selected control parameters settings. In the example shown, therapy system 12 includes NPWT device 14, sensors 16, and computing device 18.

NPWT device 14 may comprise a system for providing fluid delivery to a wound therapy dressing, e.g., NPWT dressing 20. NPWT device 14 may include a reservoir and a negative pressure source coupled to the reservoir and NPWT dressing 20. In some examples, NPWT device 14 may further include a fluid flow device in fluid communication with the fluid supply reservoir, e.g., one or more of a pump, a valve, or a generator. As used herein, a “fluid” refers to any substance that deforms or “flows” when subjected to one or more external forces (e.g., pressure, gravity, etc.). Fluids, whether in a liquid state (e.g., saline solution, distilled water, etc.), a gaseous state (e.g., a mixture of gases such as atmospheric air, a gaseous element such as pure oxygen, etc.), a plasma state, or others can be used in accordance with various techniques described herein, such as NPWT.

NPWT device 14 may be, for example, a Veraflo™ Therapy System, a V.A.C.ULTA™ Therapy System which may include INFOV.A.C.™ Canisters, a V.A.C.® Therapy System, and an ActiV.A.C.™ Therapy System from 3M™ Company of St. Paul, Minnesota. NPWT dressing 20 may be, for example, a V.A.C. VERAFLO™ Dressing, V.A.C. VERAFLO™ Large Dressing, V.A.C. VERAFLO™ CLEANSE Dressing, and a V.A.C. VERAFLO CLEANSE CHOICE™ Dressing from 3M™ Company of St. Paul, Minnesota

In some examples, NPWT device 14 may utilize a gravity fluid flow from the fluid supply reservoir to NPWT dressing 20 without utilizing a pumping device. For example, the fluid flow device of NPWT device 14 may be a valve (e.g., a solenoid-actuated pinch valve) configured to control the flow of fluid between the fluid supply reservoir and NPWT dressing 20. In some examples, the negative pressure source may draw fluid into NPWT dressing 20 from the fluid supply reservoir, e.g., without the aid of gravity feed or a pumping action from a fluid flow device.

In some examples, the negative pressure source may comprise a diaphragm vacuum pump. In some examples, NPWT device 14 may include a filter or muffler coupled to the negative pressure source, e.g., to reduce the operating noise of the negative pressure source and/or filter air exiting the negative pressure source.

The fluid flow device of NPWT device 14 may comprise a pump, e.g., a peristaltic pump, centrifugal pump, or other suitable pump. In other examples, the fluid flow device of NPWT device 14 may comprise a gravity feed system instead of (or in conjunction with) a pump to deliver fluid to NPWT dressing 20. For example, a valve between the gravity feed system and NPWT dressing 20 may be used to restrict the fluid flow to NPWT dressing 20 when a predetermined pressure is reached.

In some examples, NPWT device 14 may also include a vent on the reservoir and a check valve configured to allow flow in the direction from NPWT dressing 20 towards the negative pressure source and restrict fluid flow in the reverse direction. NPWT device 14 may also include a pressure sensor coupled to NPWT dressing 20, as well as a pressure sensor coupled to the negative pressure source and wound dressing 20.

In some examples, NPWT device 14 may operate in three modes. In a first mode, the negative pressure source may be activated to create a negative pressure on NPWT dressing 20 while the fluid flow device of NPWT device 14 is not activated. In a second mode, the negative pressure source may not be activated but the fluid flow device of NPWT device 14 may be activated to provide a fluid flow to NPWT dressing 20. In a third mode, both the negative pressure source and the fluid flow device of NPWT device 14 may not be activated.

In an example of the operation of NPWT device 14, the negative pressure source may be activated to create a negative pressure on the reservoir and NPWT dressing 20. The pressure at the negative pressure source and the reservoir and NPWT dressing 20 may be monitored via one or more pressure sensors. When the desired level of negative pressure (e.g., −125 mm Hg) is achieved, the negative pressure source may be deactivated and the vent may be opened to vent the reservoir, e.g., to atmosphere. In some examples, the check valve may maintain the negative pressure on NPWT dressing 20, which may be monitored via one or more pressure sensors. In some examples, the check valve may be a duckbill type or ball-check type or flap type valve.

The fluid flow device of NPWT device 14 may then be activated to begin fluid delivery to NPWT dressing 20. In some examples, the fluid flow device of NPWT device 14 may be configured to flow at various rates, such as approximately 70 to 90 ml/minute in the case of the fluid being a liquid with a viscosity in the general range of a saline solution, or lower in the case of certain gases, such as oxygen. As fluid is pumped from the fluid flow device of NPWT device 14 to NPWT dressing 20, the pressure at NPWT dressing 20 (which may be monitored via the one or more pressure sensors) may increase. When NPWT dressing 20 reaches a predetermined pressure, a pressure sensor (which may be used to sense both positive and negative pressures) may send a control signal to a control device of NPWT device 14 (e.g. a control switch or actuator) to restrict fluid flow from the fluid flow device of NPWT device 14 to NPWT dressing 20. The increase in pressure of NPWT dressing 20 may be used as an indication that fluid from the fluid flow device of NPWT device 14 has sufficiently filled NPWT dressing 20. By monitoring the pressure of NPWT dressing 20 with the one or more pressure sensors of NPWT dressing 20 and/or of NPWT device 14, NPWT device 14 may reduce the likelihood of NPWT dressing 20 becoming overfilled. This may reduce waste of fluid and leakage of NPWT dressing 20 associated with overfilling. It will be understood that interface circuitry (not shown purely for purposes of ease of illustration) may be utilized to generate a sufficiently strong control signal and to implement the control logic.

The fluid flow device of NPWT device 14 may be a valve (e.g., a solenoid-actuated pinch valve) that restricts fluid flow from the fluid supply reservoir or a pump that may be activated to provide fluid flow. The operation of the fluid flow device of NPWT device 14 (e.g., the position of a valve or the activation/deactivation of a pump) may be automatically altered when a predetermined pressure is reached at NPWT dressing 20. In some examples, the predetermined pressure of NPWT dressing 20 at which the operation of the fluid flow device of NPWT device 14 is altered may be approximately 1.0 mm Hg (gauge pressure as measured by a pressure sensor). In some examples, the predetermined pressure may fall between −10 and 10 mm Hg, including values of −10, −9, −8, −7, −6, −5, −4, −3, −2, −1, 0, 1, 2, 3, 4, 5, 6, 7, 8, 9 or 10 mm Hg, or any values between.

In other examples, a user may monitor one or more pressure sensors of NPWT dressing 20 and/or of NPWT device 14 and manually control operation of the fluid flow device of NPWT device 14 when NPWT dressing 20 reaches the predetermined pressure. For example, a user may deactivate the fluid flow device of NPWT device 14 by manipulating a control switch of NPWT device 14 or restrict fluid flow from the fluid flow device of NPWT device 14 by closing a valve.

When NPWT dressing 20 is sufficiently filled with fluid, the user may continue with the desired fluid instillation and vacuum therapy treatments. In some examples, NPWT device 14 and NPWT dressing 20 may be used for instillation cycling, which may offer advantages for wound dressings on articulated joints (e.g., knee) where the wound or dressing volume could be influenced by the patient's body position. In some examples, utilizing a foam NPWT dressing 20, the volume of NPWT dressing 20 may change over time in part due to compression-set of the foam. For example, the volume of the foam may be reduced over time as the foam is subjected to pressure. This change in volume occupied by the foam may affect the volume of fluid needed to fill NPWT dressing 20. In some examples, NPWT device 14 may utilize pressure readings to indicate when NPWT dressing 20 has received a sufficient volume of liquid. In some examples, NPWT device 14 may utilize absorption layers in the wound dressing instead of, or in addition to, a reservoir.

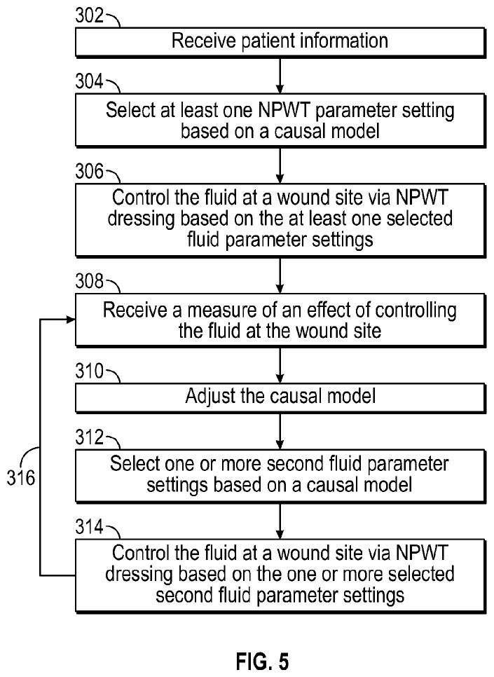

Sensor 16 may include one or more of any of a pressure sensor, a flow sensor, a resistance, current, voltage, and/or impedance sensor (such as described below with reference to FIG. 5), a temperature sensor, a humidity sensor, a biometric sensor including, but not limited to, a blood pressure sensor, a heart rate sensor, a blood glucose level sensor, an albumin and/or pre-albumin sensor, an oxygen sensor, a tissue oxygen concentration sensor, an oxygenated hemoglobin level sensor, a wound sensor, an on-body sensor, a patient activity sensor including, but not limited to, an accelerometer, a carbon dioxide sensor, a pH sensor, an analyte sensor configured to measure a biomarker, a protein, a growth factor, cytokines, foreign DNA, microbes, and the like, an optical sensor configured to measure the reflectance, transmittance, absorbance and/or opacity at one or more wavelengths of light, e.g., of a wound and/or a fluid, a viscosity sensor, a turbidity sensor, a specific gravity sensor, or any other suitable sensor.

Computing device 18 may be configured to process data and/or information from sensors 16 and/or any of user device 6 and server 24 and database 8, e.g., either directly connected or through a connection to network 10, and automatically control NPWT device 14. For example, computing device 18 may gather, collect, and/or compile patient information from a plurality of NPWT patients, including patient 4. Computing device 18 may be configured to transmit patient information, e.g., of patient 4, to user device 6, server 24, database 8, and/or any other suitable device or database.

In some examples, computing device 18 may be configured to execute therapy parameter unit 22. For example, computing device 18 may be configured to execute, after receiving patient information, therapy parameter unit 22 to determine one or more NPWT parameters and/or settings based on the patient information, e.g., one or more NPWT parameters. In other examples, computing device 18 may be configured to output patient information, e.g., of patient 4, receive one or more NPWT parameters and/or settings from another device that may execute therapy parameter unit 22, and control NPWT device 14 based on the received one or more NPWT parameters and/or settings. In other words, therapy parameter unit 22 may be executed by a different device, e.g., user device 6, server 24, or any other suitable device, and computing device 18 may be configured to send and receive patient information, such as sending patient 4 information to the other device executing therapy parameter unit 22, receive one or more NPWT parameters and/or settings, e.g., determined by therapy parameter unit 22, and control NPWT device 14 to provide NPWT in accordance with the received one or more NPWT parameters and/or settings.

Therapy parameter unit 22 may be configured to receive patient information and select at least one NPWT parameter and/or setting, e.g., for controlling a fluid at a wound site via NPWT device 14 and NPWT dressing 20. In some examples, therapy parameter unit 22 may be configured to select at least one NPWT parameter and/or setting based on a causal model that determines current causal relationships between a set of NPWT parameters and/or settings and a set of effects of controlling the fluid at the wound site, such as a Deep Causal Learning (DCL) model. In some examples, therapy parameter unit 22 may be configured to control a fluid at the wound site via NPWT dressing 20 based on the selected at least one NPWT parameter setting, receive a measure of an effect of controlling the fluid at the wound site, e.g., via one or more of sensors 16, and adjust the DCL model based on the received measure of the effect of controlling the fluid at the wound site. In some examples, the selected at least one NPWT parameter setting may include a negative pressure level, a negative pressure cycling, a continuous negative pressure application, a fluid flow rate, a fluid volume, a fluid pressure, a fluid temperature, a fluid composition, a fluid dwell time, and a fluid purge time. In some examples, controlling the fluid at the wound site may include providing the fluid to the wound site via NPWT dressing 20, e.g., via NPWT device 14. In some examples, a user may control the fluid at the wound site, e.g., via NPWT device 14 and based on information from therapy parameter unit 22 that may be displayed via one or more devices, such as user device 6.

In some examples, patient information includes at least one of user input patient information, patient biometric information, and wound measurement information, e.g., wound measurement information such as a measure of the effect of controlling the fluid at the wound site. In some examples, wound measurement information and a measure of the effect of controlling the fluid at the wound site may include at least one of an impedance measurement of wounded tissue, an oxygen measurement of a wound bed, an oxygen measurement of the fluid, a carbon dioxide measurement of the wound bed, a temperature measurement, an analyte sensor measurement, and/or an optical measurement of the wound bed. In some examples, patient information may include an aggregate of patient information of a plurality of patients, e.g., stored on database 8. In some examples, patient information includes any information and/or data from sensors 16.

User device 6 may be a company, organization, or agency server or computing device, an individual person's computing device, a clinician's computer terminal, or any suitable device suitable for communicating with therapy system 12 and/or network 10 and/or performing any of the computing functions ascribed to any computing device herein, e.g., such as computing device 18.

In some examples, network 10 may comprise a public network, such as the Internet. Although illustrated as a single entity, network 10 may comprise a combination of public and/or private networks. In some examples, network 10 may comprise one or more of a wide area network (WAN) (e.g., the Internet), a local area network (LAN), a virtual private network (VPN), or another wired or wireless communication network.

Server 24 may be configured to gather, collect, and/or compile patient information and/or execute therapy parameter unit 22, and in some examples control NPWT device 14 and/or sensors 16, e.g., remotely. Server 24 may gather, collect, and/or compile patient information from one or more user devices 6, one or more computing devices 18, or any other suitable device and/or from direct input from a clinician and/or patient 4.

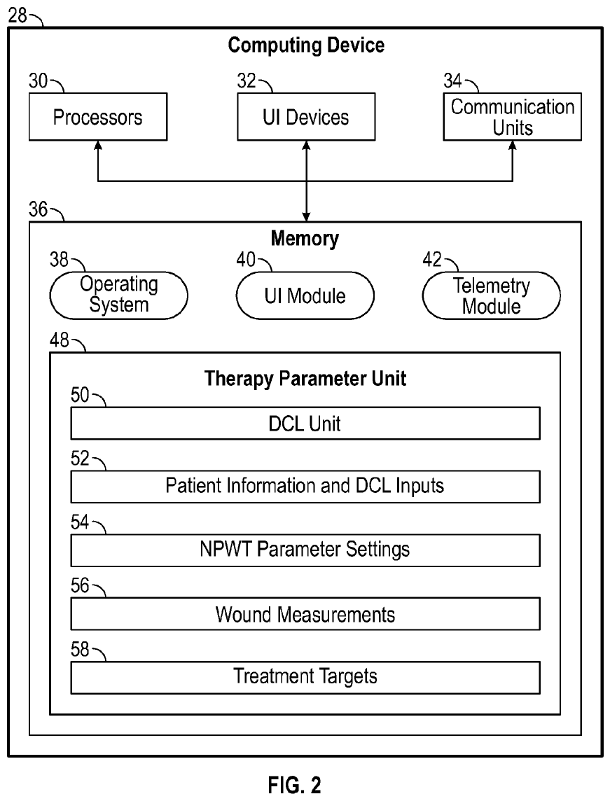

FIG. 2 is a block diagram illustrating an example computing device 28 configured to execute a causal model, in accordance with the techniques of this disclosure. Computing device 28 may be an example of server 24 of FIG. 1 or computing device 18 of FIG. 1, which may be included within or in communication with server 24 or may be a separate device from server 24. The architecture of computing device 28 illustrated in FIG. 2 is shown for exemplary purposes only and computing device 28 should not be limited to this architecture. In other examples, computing device 28 may be configured in a variety of ways.

As shown in the example of FIG. 2, computing device 28 includes one or more processors 30, one or more user interface (UI) devices 32, one or more communication units 34, and one or more memory units 36. Memory 36 of computing device 28 includes operating system 38, UI module 40, telemetry module 42, and therapy parameter unit 48, which are executable by processors 30. The various components, units, or modules of computing device 28 are coupled (physically, communicatively, and/or operatively) using communication channels for inter-component communications. In some examples, the communication channels may include a system bus, a network connection, an inter-process communication data structure, or any other system for communicating data. While shown separately in the example of FIG. 2, it will be appreciated that, in other examples, memory 36 and processors 30 may be integrated into a single hardware unit, such as a system on a chip (SoC). Whether implemented as separate components or in integrated fashion, memory 36 and processors 30 provide a computer platform for executing operation system 38. In turn, operating system 38 provides a multitasking operating environment for executing one or more software applications that computing device 28 can run.

Processors 30, in one example, may comprise one or more processors that are configured to implement functionality and/or process instructions for execution within computing device 28. For example, processors 30 may be capable of processing instructions stored by memory 36. Processors 30 may include, for example, microprocessors, a single-core processor, a multi-core processor, digital signal processors (DSPs), application specific integrated circuits (ASICs), field-programmable gate array (FPGAs), processing circuitry (e.g., fixed function circuitry, programmable circuitry, or any combination of fixed function circuitry and programmable circuitry) or equivalent discrete or integrated logic circuitry, or a combination of any of the foregoing devices or circuitry.

Memory 36 may be configured to store information (e.g., data and/or executable instructions) within computing device 28 during operation. Memory 36 may include a computer-readable storage medium or computer-readable storage device. In some examples, memory 36 may include one or more of a short-term memory or a long-term memory. Memory 36 may include, for example, one or more of random access memories (RAM), dynamic random access memories (DRAM), static random access memories (SRAM), on chip memory (e.g., in the case of SoC implementations), off chip memory, magnetic discs, optical discs, flash memories, or forms of electrically programmable memories (EPROM) or electrically erasable and programmable memories (EEPROM). In some examples, memory 36 is used to store program instructions for execution by processors 30. Memory 36 may be used by software or applications running on computing device 28 (e.g., therapy parameter unit 48) to temporarily store information during program execution.

Computing device 28 may utilize communication units 34 to communicate with external devices via one or more networks, e.g., network 10 from FIG. 1, or via wireless signals. Communication units 34 may be network interfaces, such as Ethernet interfaces, optical transceivers, radio frequency (RF) transceivers, or any other type of devices that can send and receive information. Other examples of interfaces may include Wi-Fi™, near-field communication (NFC), or Bluetooth® radios. In some examples, computing device 28 utilizes communication units 34 to wirelessly communicate with an external device, such as user devices 6 and account database 8 of FIG. 1.

UI devices 32 may be configured to operate as both input devices and output devices. For example, UI devices 32 may be configured to receive tactile, audio, or visual input from a user of computing device 28. In addition to receiving input from a user, UI devices 32 may be configured to provide output to a user using tactile, audio, or video stimuli. In one example, UI devices 32 may be configured to output content such as a graphical user interface (GUI) for display at a display device. UI devices 32 may include a presence-sensitive display that displays a GUI and receives input from a user using capacitive, inductive, and/or optical detection at or near the presence-sensitive display.

Other examples of UI devices 32 include a mouse, a keyboard, a voice-responsive system, video camera, microphone, or any other type of device for detecting a command from a user, or a sound card, a video graphics adapter card, or any other type of device for converting a signal into an appropriate form understandable to humans or machines. Additional examples of UI devices 32 include a speaker, a cathode ray tube (CRT) monitor, a liquid crystal display (LCD), organic light emitting diode (OLED), or any other type of device that can generate intelligible output to a user or a machine.

Operating system 38 controls the operation of components of computing device 28. For example, operating system 38, in one example, facilitates the communication of UI module 40, telemetry module 42, and therapy parameter unit 48 with processors 30, UI devices 32, communication units 34, and memory 36. UI module 40, telemetry module 42, and therapy parameter unit 48 may each include program instructions and/or data stored in memory 36 that are executable by processors 30. For example, therapy parameter unit 48 may include instructions that cause computing device 28 to perform one or more of the techniques described in this disclosure.

Computing device 28 may include additional components that, for clarity, are not shown in FIG. 2. For example, computing device 28 may include a battery to provide power to the components of computing device 28. Similarly, the components of computing device 28 shown in FIG. 2 may not be necessary in every example of computing device 28 that is consistent with this disclosure.

In the example illustrated in FIG. 2, therapy parameter unit 48 includes deep causal learning (DCL) unit 50, patient information and DCL inputs 52, NPWT parameter settings 54, and wound measurements 56. Therapy parameter unit 48 may be an example of therapy parameter unit 22 of FIG. 1.

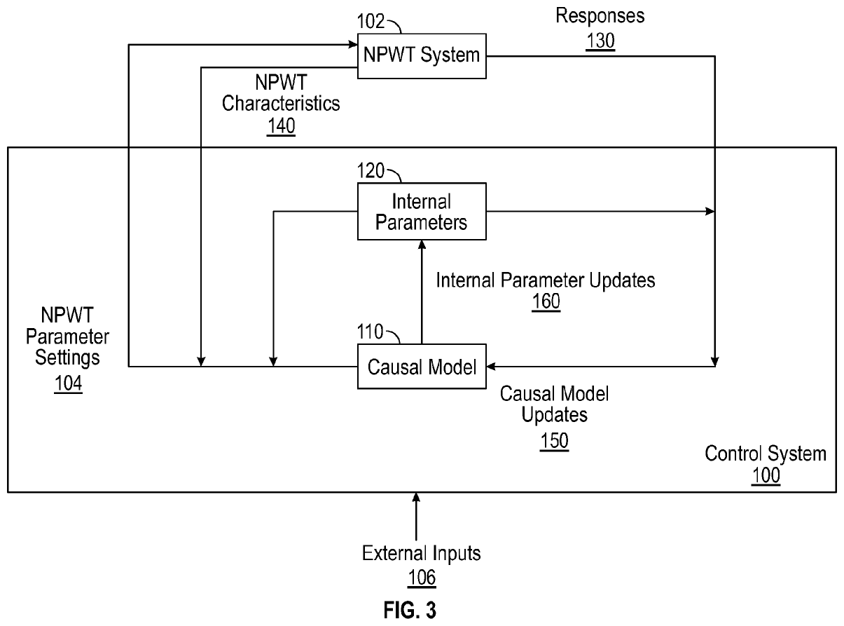

DCL unit 50 may be configured to execute a control system, such as control system 100 of FIG. 3 described below, that selects NPWT parameter settings 54 that are applied to a NPWT system, e.g., NPWT device 14 and NPWT dressing 20. In some examples, DCL unit 50 may be configured to execute the control system based on a causal model, e.g., causal model 110 and/or 210 described below with reference to FIGS. 3 & 4. DCL unit 50 may be configured to receive patient information and determine one or more NPWT parameters and/or settings based on the patient information, e.g., one or more NPWT parameters. In some examples, DCL unit 50 may be configured to receive patient information and select at least one NPWT parameter and/or setting, e.g., for controlling a fluid at a wound site via NPWT device 14 and NPWT dressing 20 of FIG. 1. DCL unit 50 may be configured to select at least one NPWT parameter and/or setting based on a causal model that determines current causal relationships between a set of NPWT parameters and/or settings and a set of effects of controlling the fluid at the wound site. In some examples, DCL unit 50 may be configured to receive a measure of an effect of controlling the fluid at the wound site, e.g., via one or more of sensors 16, and adjust the DCL model based on the received measure of the effect of controlling the fluid at the wound site. In some examples, the selected at least one NPWT parameter setting may include a negative pressure level, a negative pressure cycling parameter (in the case of instillation therapy), a continuous negative pressure application, a fluid flow rate, a fluid volume, a fluid pressure, a fluid temperature, a fluid composition, a fluid dwell time (in the case of instillation therapy), and a fluid purge time (in the case of instillation therapy).

In some examples, DCL unit 50 may be software, hardware, or a combination thereof configured to execute the causal model, e.g., via execution of DCL unit 50 by a computing device such as computing device 28, computing device 18, user device 6, server 24, or any suitable computing device. DCL unit 50 may be configured to select at least one NPWT parameter and/or setting based on the received patient information and/or other DCL inputs, e.g., patient information and/or additional DCL inputs may be utilized by the causal model during an execution phase and/or to update the causal model. Updating the causal model is part of the execution phase, i.e. as DCL is running/executing, experiments either contribute to improving the model (explore) or the patient outcome (exploit).

In some examples, DCL unit 50 may be configured to determine and/or measure current causal relationships between NPWT parameter settings 54 and wound measurements 56 and treatment targets 58, e.g., via executing a DCL model such as DCL model 110 and/or 210 described below with respect to FIGS. 3 & 4. By way of contrast, computing systems and/or software configured according to conventional ML and/or Al models and/or algorithms, or other models and/or algorithms, e.g., classification, regression, dimensionality reduction, and/or clustering ML models, rely on determining correlations between parameters and responses but do not measure causal effects and uncertainties regarding causal effects as DCL unit 50 is configured to do, in some examples. For example, DCL unit 50 may be configured to determine and/or measure impact measurements between parameter settings and responses and/or estimate of the true mean effects of parameter settings and confidence intervals for the impact measurements that represent the current level of uncertainty about causal effects, such as described below with reference to causal model 110 of FIG. 3.

In some examples, patient information and DCL inputs 52 may include patient health record data such as demographic information, e.g., age, gender, ethnicity, weight, body mass index, and the like, personal and/or family medical histories and co-morbidities, past and current diagnoses, e.g., diabetes, obesity, cardiovascular disease, cholesterol, blood pressure, and the like, prescribed medications including dosage and frequency of use, blood lab results and values, genetic test results, allergies and allergy test results, and any other suitable patient health record data. Patient information and DCL inputs 52 may include situational inputs from a clinician or the patient such as current symptoms, results from previous and most recent wound inspections, e.g., wound images over time, wound geometry measurements (length, width, depth, volume) over time, infections and suspected infections, infection history and lab results regarding culture count and speciation over time, frequency and history of wound dressing changes, NPWT treatment constraints (e.g., clinician determined minimum and/or maximum negative pressure, and the like), discomfort metrics (e.g., pain scores and/or ratings over time, pain history associated with dressing changes), patient behavior schedule (e.g., rest/sleep schedule, work schedule, activity/exercise schedule), and the like. Patient information and DCL inputs 52 may include any applicable and established prognosis models, such as the current confidence level of a clinician and/or patent regarding the current treatment.

Patient information and DCL inputs 52 may further include measured patient biometrics such as real-time measurement and record of patient vital information, e.g., temperature, heart rate, blood pressure, systemic blood oxygenation, and/or any data/information from sensors 16, and patient activity via measurement, e.g., movement/accelerometer data which may measure/detect rest/sleep times, patient itching and/or scratching at or near the wound and/or dressing. Patient information and DCL inputs 52 may include wound related measurements such as real-time and historical measurement of the wound and/or wound bed, e.g., impedance measurements and spatial mapping of the wound (which may indicate granulation tissue thickness, degree of epithelial coverage, current healing stage such as inflammation, proliferation, remodeling, etc., healing stage transition, healing progress, healing stall, and the like), measurements of wound oxygen, carbon dioxide, temperature, pH, and the like, analyte sensor measurement such as biomarkers, proteins, growth factors, cytokines, foreign DNA, and optical measurements such as reflectance and/or absorbance spectra and/or at one or more wavelengths of the wound bed. Patient information and DCL inputs 52 may include real-time and historical measurements about and/or near the wound bed such as impedance measurements and spatial mapping across the wound bed, e.g., which may indicate wound bed depth, impedance measurements and spatial mapping about circumferential segments of the wound bed, e.g., peri-wound impedance mapping and measurements which may detect subdermal features such as tunneling and undermining and their severity (e.g., a relative size of a void space from tunneling and/or undermining) and/or maceration, and peri-wound optical measurements such as reflectance and/or absorbance spectra and/or at one or more wavelengths.

In some examples, patient information and DCL inputs 52 may further include real-time and historical exudate and/or instillation fluid collection vessel measurements such as compositional measurements of ionic strength and/or presence of analytes (e.g., biomarkers, proteins, growth factors, cytokines, foreign DNA, etc.), oxygen, carbon dioxide, temperature, pH measurements, viscosity, turbidity, and/or specific gravity of collected liquid, optical measurements indicating liquid transparency and/or opacity, and optical measurements related to reagent-based assays, e.g., optical absorbance, fluorescence, luminescence measurements, etc.

In some examples, patient information and DCL inputs 52 may further include any patient information, measurements, and data aggregated from a plurality of patients. Patient information and DCL inputs 52 may further include criteria for classifying “raw measured data,” e.g., current, impedance, voltage of a sensor/and or detector, etc. Patient information and DCL inputs 52 may further include criteria for ranking the importance of data included in patient information and DCL inputs 52.

NPWT parameter settings 54 may include any parameter setting that may be regulated in NPWT and irrigation/instillation wound treatment systems, e.g., NPWT device 14 and NPWT dressing 20. For example, NPWT parameter settings 54 may include negative pressure level, negative pressure cycling (in the case of instillation) and/or “on” times and “off” times (in the case of instillation), duration and negative pressure levels (during instillation intervals or on a continuous basis for irrigation) of one or more pressure schedules and/or regimens. In some instillation-based scenarios, NPWT parameter settings 54 may include a stepwise or discrete pressure schedule including differing negative pressures for the same and/or differing durations. NPWT parameter settings 54 may include a negative pressure vs. time waveform, e.g., for a continuous pressure schedule in the case of irrigation therapy, such as a sinusoidal waveform, a triangular waveform, a square-wave waveform, or any other waveform. NPWT parameter settings 54 may include negative pressure pump down and pump up times, e.g., such as ramping rates, overall length of therapy, and time between dressing changes. NPWT parameter settings 54 may include instillation parameters such as fluid delivery fill time and volumetric rate, fluid volume, fluid pressure, fluid dwell time, fluid purge time and volumetric rate, fluid composition (e.g., ionic strength and electrolytic composition, antimicrobials, chemical curation agents, antiseptics, anti-astringents, hormones, cytokines, anti-inflammatories, immune response activators, immune response inhibitors, presence and dose of gases such as oxygen, carbon dioxide, and the like), fluid fill, dwell, and draw temporal profile (e.g., higher-frequency dynamics that may loosen and/or disrupt sluff, biofilm, ingrowth of tissue into the dressing, a fill/purge duty cycle and or waveform such as a sinusoidal waveform, a triangular waveform, a square-wave waveform, or any other waveform), and the like.

In some examples, therapy parameter unit 48 may include one or more NPWT treatment targets or objectives, e.g., treatment targets 58. For example, DCL unit 50 may control and automate a multivariate, dynamic system to improve and/or optimize multiple treatment targets 58. In some examples, DCL unit 50 may improve and/or optimize a plurality of treatment targets 58 in parallel and/or simultaneously, e.g., for one or more patients.

Treatment targets 58 may include rapid granulation, rapid epithelialization, reduced wound healing stalling, reduced occurrence of tunneled and undermined areas, rapid shrinking of tunneled and undermined areas while reducing abscess formation, reduced biofilm or infection, improved quality of regenerated tissues, improved graft integration, reduced dressing tissue ingrowth, reduced patient pain and discomfort, improved patient sleep and restfulness, reduced dressing changes (e.g., only change when needed), and reduced peri-wound maceration.

In some examples, causal models and/or DCL algorithms, e.g., DCL unit 50, may enable learnings from a given patient to be transferrable to subsequent patients, as opposed to other machine learning (ML) and/or artificial intelligence (AI) algorithms, models, or schemes. Causal models and/or DCL algorithms such as DCL unit 50 may determine cause and effect relationships between NPWT parameter settings 54, wound measurements 56, and treatment targets 58. By way of contrast, other ML and/or Al algorithms may determine correlations between NPWT parameter settings 54, wound measurements 56, and treatment targets 58, and may not determine correct NPWT parameter settings 54 as quickly (e.g., in fewer iterations) or as accurately in order to cause the desired treatment targets 58 to occur. For example, other ML and/or Al algorithms may not determine and/or measure current causal relationships between NPWT parameter settings 54 and wound measurements 56 and treatment targets 58, such as described below with reference to FIG. 3 and NPWT settings 104 and responses 130. As such, other ML and/or Al algorithms may spend computing time and/or resources exploring “weaker” correlations, e.g., correlations without a causal relationship and/or with a low causal impact between NPWT parameter settings 54 and wound measurements 56 and treatment targets 58, largely because other ML and/or Al algorithms do not determine, know, and/or measure causal relationships but rather rely on exploring determined correlations. For example, other ML and/or Al algorithms may not be configured to isolate causation from historical data and may not be configured to quantify relationships with the precision required to drive decision making, e.g., that fact that a particular NPWT parameter setting is correlated with a wound measurement may not be sufficient to recommend changing that particular NPWT parameter or recommending a change by a specific amount. The higher the dimensionality of a problem, e.g., determining NPWT parameter settings 54, and the number of features, the more likely spurious correlations are to happen, making it that much more difficult for any algorithm to identify the key variables, e.g., of NPWT parameter settings 54, that actually and reliably drive desired outcomes.

In some examples, causal models and/or DCL algorithms such as DCL unit 50 may aggregate learning from a plurality of patients which may increase statistical power, and DCL unit 50 may create smaller aggregates/clusters representing sub-populations over time, and adjust NPWT parameter settings 54 tailored to the sub-populations. In some examples, causal models and/or DCL algorithms such as DCL unit 50 may determine which contextual data lead to a different course of action, e.g., different NPWT parameter settings 54, for a particular patient, e.g., personalized medicine.

FIG. 3 is a conceptual diagram illustrating a control system 100 that selects NPWT parameter settings 104 that are applied to a NPWT system 102, in accordance with one or more techniques of this disclosure. Each of NPWT parameter settings 104 defines a setting for each of multiple controllable elements of the NPWT system 102, and may be substantially the same as NPWT parameter setting 54. Generally, the controllable elements of NPWT system 102 are those elements that can be controlled by the system 100 and that can take multiple different possible settings. Control system 100 may be performed and/or executed, for example, by DCL unit 50 described above.

During operation, the control system 100 may repeatedly select NPWT parameter settings 104 and monitor responses 130 to the control settings 104. Responses 130 may be measured via sensors 16. For example, a sensor 16 may measure an impedance of a wound, a sensor 16 may measure a blood pressure, and a sensor 16 may measure a biomarker, and the measurements may be responses 130.

The system 100 can compute a performance metric based on responses 130, e.g. can compute a range of values that includes one or more single values that represent the performance of the system in controlling the NPWT system 102 to improve and/or maximize the quality and/or efficacy of the NPWT delivered. A measure of quality used by control system 100 may be one or more predetermined values for responses 130. An example performance metric that combines all of the measures of quality used by the system is a weighted sum of the values of the chosen measures of quality.

As another example, the performance metric can be a weighted sum of, for each of the measures of quality, a difference between the measure of quality and a baseline or desired value for the measure of quality, e.g., so that the system tries to reduce and/or minimize deviation outside of acceptable values for each of the measures of quality. For example, the performance metric may be a weighted sum of a difference between responses 130 and treatment targets 58. Another example of such a performance metric is a weighted sum of, for each of the measures of quality, a function that is zero if the measure of quality is within an acceptable range, and is equal to the distance from the measure of quality to the closest end point of the acceptable range if the measure of quality is outside the acceptable range.

Control system 100 may also monitor the NPWT characteristics 140 of NPWT system 102. Generally, the NPWT characteristics 140 can include any data characterizing the NPWT system 102 that may modify the effect that NPWT parameter settings 104 have on responses 130 but that are not accounted for in the NPWT parameter settings 104, e.g., that are not controllable by the control system 100. For example, the NPWT characteristics 140 of NPWT system 102 can include environmental conditions during treatment, e.g., the ambient humidity and/or ambient temperature of the environment in which the patient is located during treatment. In some cases, these measures might be controllable by NPWT system 102, e.g. NPWT system 102 may control ambient humidity and temperature. In these cases, the adjustable measures would be included as NPWT parameter settings 104 rather than NPWT characteristics 140.

Control system 100 may use the responses 130 to update a causal model 110 that models causal relationships between NPWT parameter settings 104 and the responses 130, e.g., that models how different settings for different elements affect values of responses 130, rather than or in addition to there being a correlation between NPWT parameter settings 104 and responses 130. In particular, causal model 110 measures, for each NPWT parameter setting 104 of NPWT system 102 and for each different type of response 130, the causal effects of the different possible NPWT parameter settings 104 on responses 130 and the current level of uncertainty of the control system 100 about the causal effects of the possible NPWT parameter settings 104. As a particular example, causal model 110 may include, for each different possible NPWT parameter settings 104 of a given controllable element and for each different type of response 130, an impact measurement that represents the impact of the possible NPWT parameter settings 104 on the responses 130 relative to the other possible NPWT parameter settings 104 for the controllable element, e.g., an estimate of the true mean effect of the possible NPWT parameter settings 104, and a confidence interval, e.g., a 95% confidence interval, for the impact measurement that represents the current level of control system 100 uncertainty about the causal effects.

In some examples, control system 100 computes confidence intervals that specify, for example, the 95% upper and lower bound of the impact of NPWT parameter settings 104 on system performance, e.g., as indicated by responses 130. Specifically, this allows control system 100 to identify when the selection of different NPWT parameter settings 104 results in (clinically) significant or insignificant differences.

If there is not a causal relationship between one or more NPWT parameter settings 104 and one or more responses 130, then NPWT parameter settings 104 that do not form a causal relationship to changes in the responses 130 may not be updated or changed during testing, e.g., control system 100 may refrain from testing controllable elements that do not result in significant differences. For example, to the extent that the upper and lower bounds of the confidence intervals of one or more particular NPWT parameter settings 104 indicate that even the largest effects would not result in a clinically meaningful difference, and to the extent that there is a cost to continuing to test/explore the one or more particular NPWT parameter settings 104, control system 100 may remove and/or indicate that those one or more particular NPWT parameter settings 104 may be removed from further exploration/experimentation/iteration. For example, to the extent that there is any cost, e.g., health-related, time, money, power, or otherwise, on continuing to experiment/iterate on particular one or more NPWT parameter settings 104, control system 100 may stop and/or indicate to stop experimenting/iterating on those particular NPWT parameter settings 104 because the cost may exceed any benefit, e.g., of controlling NPWT system 102 to improve performance metrics and/or to determine further causal relationships between NPWT parameter settings 104 and responses 130.

By way of contrast, conventional ML and/or Al models and/or algorithms, or other models and/or algorithms, may be correlation-based models rather than causal models. For example, classification, regression, dimensionality reduction, and/or clustering ML models rely on determining correlations between parameters and responses, but do not measure causal effects and uncertainties regarding causal effects, e.g., such as impact measurements between parameter settings and responses and/or an estimate of the true mean effects of parameter settings and confidence intervals for the impact measurements that represent the current level of uncertainty about causal effects.

In some examples, prior to beginning to control NPWT system 102, control system 100 may receive external inputs 106. External inputs 106 may include data received by the control system 100 from any of a variety of sources. For example, external inputs 106 may include data received from a user of control system 100, data generated by another control system that was previously controlling NPWT system 102, data generated by a machine learning model, data generated by one or more sensors 16, or some combination of these.

Generally, external inputs 106 specify at least initial possible values for the settings of the controllable elements of NPWT system 102 (e.g., NPWT parameter settings 104) and which responses 130 control system 100 tracks during operation.

For example, external inputs 106 may cause control system 100 to track measurements for certain sensors 16 of NPWT system 102, a performance metric such as a figure of merit, or any other objective function that is derived from certain sensor measurements, to be improved and/or optimized by control system 100 while controlling NPWT system 102, or both.

Control system 100 may use external inputs 106 to generate initial probability distributions (“baseline probability distributions”) over the initial possible setting values for the controllable elements, e.g., NPWT parameter settings 104. By initializing these baseline probability distributions using external inputs 106, control system 100 may constrain NPWT parameter settings 104 selections to those which do not violate any constraints imposed by the external data 106 and, optionally, NPWT parameter settings 104 selections that do not deviate from historical ranges for NPWT parameter settings 104 that have previously been used to control NPWT system 102. For example, if there are certain ranges of NPWT parameter settings 104 that are known to be detrimental, then external inputs 106 may define those ranges so that control system 100 does not select NPWT parameter settings 104 within those certain ranges.

Control system 100 may also use external inputs 106 to initialize a set of internal parameters 120, e.g., to assign baseline values to internal parameters 120. Generally, internal parameters 120 define how control system 100 selects NPWT parameter settings 104 given the current causal model 110, e.g., given the current causal relationships that have been determined by control system 100 and the system uncertainty about the current causal relationships. Internal parameters 120 may also define how control system 100 updates the causal model 110 using received responses 130.

Control system 100 may update at least some of internal parameters 120 while updating the causal model 110. That is, while one or more of internal parameters 120 may be fixed to the initialized, baseline values during operation of control system 100, control system 100 may repeatedly adjust others of the internal parameters 120 during operation in order to allow control system 100 to more effectively measure and, in some cases, exploit causal relationships. For example, in order to control NPWT system 102 during operation, control system 100 may repeatedly identify procedural instances within NPWT system 102 based on internal parameters 120.

Each procedural instance may be a collection of one or more entities within NPWT system 102 that may be associated with a time window. An entity within the NPWT system 102 may be a subset, e.g., either a proper subset or an improper subset, of NPWT system 102. In particular, an entity is a subset of the NPWT system 102 for which responses 130 may be obtained and which may be impacted by applied control settings, e.g., NPWT parameter settings 104. For example, when NPWT system 102 includes multiple physical entities from which sensor 16 measurements may be obtained, a given procedural instance may include a proper subset of the physical entities to which a set of NPWT parameter settings 104 may be applied. The number of subsets into which the entities within NPWT system 102 may be divided may be defined by internal parameters 120

How control system 100 divides the entities into subsets at any given time during operation of control system 100 may be defined by internal parameters 120 that define the spatial extent of NPWT parameter settings 104 applied by control system 100 for the instance. The spatial extent of an instance identifies the subset of the response 130 that may be assigned to the instance, e.g., such that responses 130 that are obtained from that subset will be associated with the instance.

For example, a procedural instance may include one or more machines that operate using NPWT parameter settings 104. The spatial extent may define the number and type of machines of the procedural instance. Control system 100 may obtain the responses 130 to selected NPWT parameter settings 104 for the given group of machines. For example, as described above, control system 100 may select NPWT parameter settings 104 related to one or more measurements of one or more sensors 16, and then control system 100 may track selected performance metrics of NPWT treatment using the selected NPWT parameter settings.

The length of the time window associated with the entities in any given procedural instance may be further defined by internal parameters 120. In particular, the time window that control system 100 may assign to any given procedural instance may be defined by internal parameters 120 that define the temporal extent of NPWT parameter settings 104 applied by control system 100. This time window, e.g., the temporal extent of the instance, may define future responses 130 that control system 100 may determine were caused by NPWT parameter settings 104 that were selected for the procedural instance.

Because internal parameters 120 may change during operation of control system 100, the instances generated by system 100 may also change. That is, control system 100 may modify how the procedural instances are identified as control system 100 changes internal parameters 120. Control system 100 may then select NPWT parameter settings 104 for each instance based on internal parameters 120 and, optionally, on NPWT characteristics 140.

In some examples, such as when control system 100 may be exploring a space of possible NPWT parameter settings 104, control system 100 may select NPWT parameter settings 104 for all of the instances based on the baseline probability distributions. In other examples, such as when control system 100 is exploiting causal relationships that have already been determined to improve and/or optimize an objective function, control system 100 may select NPWT parameter settings 104 for some of the instances (e.g., “hybrid instances”) using the current causal model 110 while continuing to select NPWT parameter settings 104 for others of the instances (e.g., “baseline instances”) based on the baseline probability distributions. More specifically, at any given time during operation of control system 100, internal parameters 120 may define the proportion of hybrid instances relative to the total number of instances. Control system 100 may also determine, for each instance, which responses 130 may be associated with the instance, e.g., for use in updating causal model 110, based on internal parameters 120.

Control system 100 may then select and/or set NPWT parameter settings 104 for each of the instances and monitor responses 130. Control system 100 may map responses 130 to impact measurements for each instance and use the impact measurements to determine causal model updates 150 that may be used to update the current causal model 110. Control system 100 may determine, based on internal parameters 120, which historical procedural instances (and responses 130 associated with the instances) may be considered by the causal model 110, and may determine causal model updates 150 based only on these determined historical procedural instances. A set of internal parameters 120 that define a data inclusion window may determine which historical procedural instances are considered by causal model 110. The data inclusion window may specify, at any given time, one or more historical time windows during which a procedural instance occurred in order for the results for that procedural instance, e.g., responses 130 associated with that procedural instance, to be considered by the causal model 110. Updating causal model 110 may be part of the execution phase, i.e. as causal model 110 is running and/or executing, and experiments may contribute to improving the model (explore) or the patient outcome (exploit).

For those internal parameters 120 that are being varied by control system 100 during operation, control system 100 may periodically generate internal parameter updates 160, e.g., to update internal parameters 120 being maintained by control system 100, based on causal model 110. In other words, as the causal model 110 changes during operation of control system 100, control system 100 may also update internal parameters 120 to reflect the changes in causal model 110. In some example, such as when control system 100 assigns some NPWT parameter settings 104 to exploit the current causal model 110, control system 100 may also use the difference between system performance for “hybrid” instances and “baseline” instances to determine internal parameter updates 160.

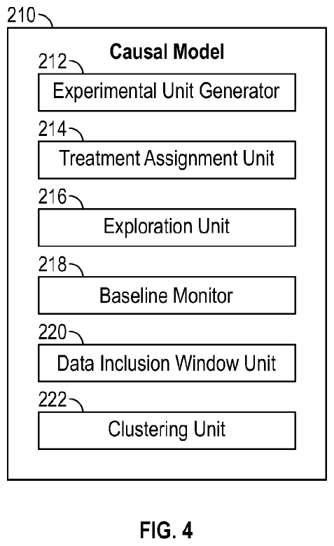

FIG. 4 is conceptual diagram illustrating an example causal model 210, in accordance with one or more techniques of this disclosure. Causal model 210 may be an example of causal model 110 and may be executed by hardware and/or software, such as DCL unit 50. In some examples, causal model 210 includes a number of processes, and in other examples causal model 210 may include other processes, data, inputs, outputs, feedback, interactions, and the like. In the example shown, causal model 210 includes experimental unit generator 212, treatment assignment unit 214, exploration unit 216, baseline monitor 218, data inclusion window unit 220, and clustering unit 222.

Experimental unit generator 212 may be configured to generate spatial-temporal experimental units. An experimental unit may be the smallest spatial-temporal extent that prevents carryover effects from degrading the causal knowledge generated, e.g., current causal relationships between parameters, such as NPWT parameter settings 104 and/or 54, and a set of effects of controlling a system, such as NPWT system 102 and/or therapy system 12. In some examples, in order to buffer carryover effects from previous experimental units, experimental unit generator 212 may limit causal model data recording to the second half of the temporal extent of each experimental unit. In addition, for each independent variable (e.g., which may be one or more NPWT parameter settings 104 and/or 54), experimental unit generator 212 may systematically vary and/or explore the spatial-temporal extent of experimental units, initially within minimum and maximum of temporal-spatial range and or constraint data, to determine an improved and/or optimal experimental unit size corresponding to a mean effect size that lies at the 95% confidence interval (p=0.05) from an asymptotic mean effect for large spatial-temporal extents.

Treatment assignment unit 214 may assign treatments to experimental units. For example, treatment assignment unit 214 may assign treatments to experimental units according to predetermined procedures, such as randomizing without replacement and counterbalancing. Treatment assignment unit 214 may also assign independent variable levels to stochastically equivalent experimental units with the constraint that the relative frequency of assignment matches the relative frequency specified by one or more exploration and/or exploitation trade-offs. In some examples, treatment assignment unit 214 may compute D-scores and confidence intervals around D-scores for each independent variable level, e.g., by taking the difference between the mean effect when “on” and the mean effect when “off” over a data inclusion window (described below) so as to provide unbiased estimates of the causal effect of the independent variable level/treatment assignment on a utility function.

Exploration unit 216 may determine whether to allocate an experimental unit toward making the most probabilistically optimal decision and/or toward improving the precision of a probability estimate. For example, exploration unit 216 may determine whether to allocate an experimental unit by probability matching. In some examples, exploration unit 216 may vary the aggressiveness of an explore/exploit ratio and control the explore/exploit ratio to determine the aggressiveness that improves and/or maximizes utility (e.g., including reducing and/or minimizing regret), such as measured and/or monitored by baseline monitor 218 (described below). In some examples, if a cost (including opportunity cost) of executing treatments is non-uniform across independent variable levels, Bonferroni-corrected confidence intervals may be computed such that more evidence is required to exploit more expensive treatments.

Baseline monitor 218 may determine, e.g., via statistical power analysis, the number of baseline experimental units needed to monitor the difference in performance between baseline trials and treatment assignments. Baseline monitor 218 may assign independent variable levels randomly sampled according to the normative operational range data to baseline experimental units. The difference between the baseline trials and the explore/exploit trials may provide an unbiased measure of utility of causal model 210 internal parameters (e.g., clustering, data inclusion window, explore/exploit aggressiveness), which may allow parameters to be objectively tuned. The baseline trials may also enable exploration of the entire search range defined by hard constraints.

Data inclusion window unit 220 may use a factorial analysis of variance (ANOVA) on blocked time ranges to analyze the impact of the blocked time ranges on the stability of the strength and direction of interactions between independent variables and the utility function. For example, for each independent variable, data inclusion window unit 220 may identify a pareto optimum data inclusion window that improves and/or maximizes both experimental power (across all experimental unit clusters and the entire decision search space) and statistical significance of causal effects. In some examples, such an inclusion window may prevent causal model 210 from over-fitting data and allows causal model 210 to remain highly responsive to dynamic changes in the structure of the underlying system.

Clustering unit 222 may manage dimensionality, e.g., whereby causal model 210 learns how to conditionally assign independent variable levels based on the factorial interactions between their effects and the attributes of the experimental units that cannot be manipulated by the control system (e.g., sex, age, comorbidity, weather, demand, etc.). Clustering unit 222 may pool experimental units into clusters of maximum within-cluster similarity of the impact of independent variables on utility and of maximum between-cluster difference. In some examples, clustering unit 222 may use a factorial ANOVA to find the factors that explain the largest amount of variance between clusters, and stepwise statistical power analysis may be used to select a number of factors that results in clusters with sufficient statistical power to find exploitable effects. In some examples, clustering unit 222 may control clustering decisions by continuously testing the clustering decisions and using baseline monitoring to objectively explore and exploit the clustering decisions impact on utility.

FIG. 5 is a flowchart of an example method of selecting at least one NPWT parameter of a wound therapy system, in accordance with one or more techniques of this disclosure. Although FIG. 5 is discussed using computer-based system 2 of FIG. 1, computing device 28 of FIG. 2, and control system 100 of FIG. 3, it is to be understood that the methods discussed herein may include and/or utilize other systems and methods in other examples.

Computing device 28 may receive patient information (302). For example, computing device 28 may receive patient information and DCL inputs 52. In some examples, patient information may include user input patient information, patient biometric information, an impedance measurement of wounded tissue, an oxygen measurement of a wound bed, an oxygen measurement of a fluid, a carbon dioxide measurement of the wound bed, a temperature measurement, an analyte sensor measurement, or an optical measurement of the wound bed each of which may be of an individual patient or an aggregate of a plurality of patients.

DCL unit 50 may select at least one NPWT parameter setting 54 and/or 104 for controlling a fluid at a wound site via NPWT dressing 20 based on a causal model, e.g., causal model 110, that determines current causal relationships between a set of NPWT parameter settings 54 and/or 104 and a set of effects of controlling the fluid at the wound site (304). In some examples, the causal model, e.g., causal model 110, may measure current causal relationships between a set of NPWT parameter settings 54 and/or 104 and a set of effects of controlling the fluid at the wound site, e.g., via measuring the causal effects of the different possible NPWT parameter settings 104 on responses 130 and the current level of uncertainty of the control system 100 about the causal effects of the possible NPWT parameter settings 104, as described above with reference to FIG. 3. As used herein, a “fluid” refers to any substance that deforms or “flows” when subjected to one or more external forces (e.g., pressure, gravity, etc.). Fluids, whether in a liquid state (e.g., saline solution, distilled water, etc.), a gaseous state (e.g., a mixture of gases such as atmospheric air, a gaseous element such as pure oxygen, etc.), a plasma state, or others can be used in accordance with various techniques described herein, such as NPWT.

Computing device 28 may control the fluid at the wound site via the NPWT dressing 20 based on the selected at least one NPWT parameter setting 54 and/or 104 (306). For example, computing device 28 may cause NPWT device 14 to operate according to the at least one selected NPWT parameter setting 54 and/or 104.

Computing device 28 and DCL unit 50 may receive a measure of an effect of controlling the fluid at the wound site (308). For example, DCL unit 50 may receive one or more measurements from one or more sensors 16, such as an impedance measurement system that may be included with NPWT wound dressing 20 or otherwise included at the wound site to measure the impedance of the wound.

DCL 50 may adjust the causal model (310). For example, DCL 50 may generate causal model updates 150 and internal parameter updates 160 to adjust the causal model based on the received measure of the effect of controlling the fluid at the wound site to update. DCL 50 may update and/or adjust the width of confidence intervals of the estimates of cause and effects, e.g., the upper and lower bounds of the impact of NPWT parameter settings 104 on system performance.

DCL unit 50 may select at least one second NPWT parameter setting 54 and/or 104 for controlling a fluid at a wound site via NPWT dressing 20 (312). For example, the previously selected NPWT parameter setting 54 and/or 104 may include a plurality of setting selections, and the second NPWT parameter setting 54 and/or 104 may be a different parameter which also may include a plurality of setting selections. In some examples, DCL unit 50 may select the at least one second NPWT parameter setting 54 and/or 104 based on the causal model, e.g., the adjusted and/or updated causal model 110, which may determine current causal relationships (which may be different than before adjustment) between a set of NPWT parameter settings 54 and/or 104 and a set of effects of controlling the fluid at the wound site.

Computing device 28 may control the fluid at the wound site via the NPWT dressing 20 based on the selected at least one second NPWT parameter setting 54 and/or 104 (314). Computing device 28 may then repeat steps (308)-(314), e.g., to continue both adjusting and/or updating causal model 110 and controlling NPWT system 102 via selecting NPWT parameter settings 54 and/or 104 based on the adjusted and/or updated causal model 110 (316).

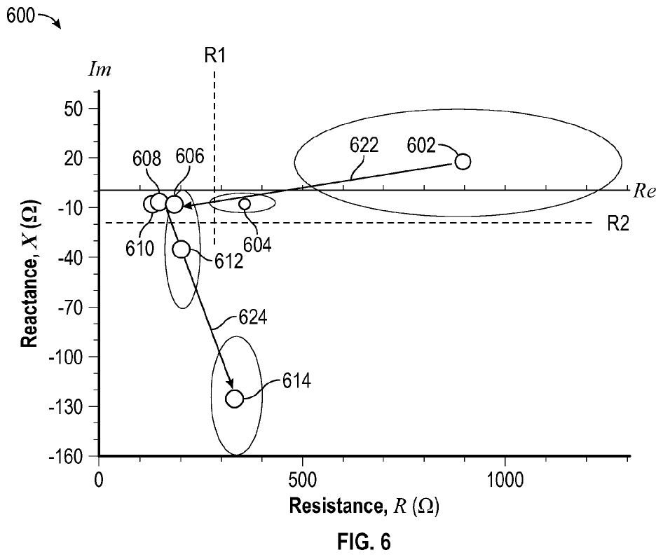

FIG. 6 is a plot 600 of example complex impedances of a tissue site at a predetermined frequency F1 measured at a plurality of times after a wound occurs at the tissue site. The example shown in FIG. 6 illustrates a progression of the impedance of a tissue site at a predetermined frequency F1 as a wound progresses through multiple stages of healing. In the example shown, the impedance measurements 602-612 correspond to the average value of the impedance measurements at the eight wounded tissue sites at frequency F1 and corresponding to the six times after the occurrence of the wound, e.g., days 0, 3, 7, 10, 14, and 16. Plot 600 is a resistance vs. reactance plot in which each mean impedance measurement value 602-612, e.g., averaged over the eight wounded tissue sites, is represented by a resistance (i.e., the real component of its complex impedance) and reactance (i.e. the imaginary component of its complex impedance) value pair. In other words, plot 600 is a two-dimensional (2D) plot of the mean impedance measurements at the different times, and the dimensions of plot 600 are resistance and reactance. In the examples shown, the mean impedance measurements 602-612 are the values at the frequency F1, e.g., 85 kHz.

In the example shown, the progression of the impedance vs. time on plot 600 follows “paths” 622 and 624, e.g., changes in the resistance and reactance values analogous to a vectors in the 2D resistance-reactance plane. Paths 622 and 624 may correspond to changes in impedance of a wounded tissue site as the wounded tissue progresses through different stages of healing. In the example shown, a wounded tissue site may begin, e.g., around the time of the occurrence of the wound, by exhibiting no tissue growth in stage 1 (inflammatory), and the electrical characteristics of the wounded tissue may be primarily resistive with little reactance. The resistance/reactance values of impedance measurements 602 and 604 at day 0 and day 3 from the occurrence of the wound, respectively, may comprise information indicative of the wounded tissue site being in the first stage, e.g., the inflammatory stage of healing.

As the wound site heals and progresses to stage 2 (proliferation, e.g., granulation), the impedance may “follow” path 622 in which the reactance remains close to zero and the resistance reduces. In other words, the wounded tissue site may change from substantially resistive to an increased conductivity during stage 1. In some examples, a transition from stage 1 to stage 2 may be determined based on a change in resistance of the wounded tissue site to less than a threshold value R1. In the example shown, the resistance value of impedance measurement 606, corresponding to day 7, is less than threshold resistance R1 and has a reactance close to zero, indicating that the wounded tissue site has progressed from stage 1 to stage 2. In some examples, the impedance of the wounded tissue site may remain relatively conductive during stage 2. In the example shown in FIG. 6, the values of impedance measurements 606, 608 and 610, corresponding to day 7, day 10 and day 14, respectively, have relatively low resistance values and reactance values close to zero, e.g., relatively close to the origin in plot 600. The resistance/reactance values of impedance measurements 606, 608 and 610 may comprise information indicative of the wounded tissue site being in the second stage, e.g., the proliferation stage of healing.

As the wounded tissue site progresses in healing to stage 3, e.g., remodeling/re-epithelialization, the impedance of the wounded tissue site may “turn” away from the origin to increasingly negative reactance values. For example, impedance measurements of the wounded tissue site may follow path 624 which changes in direction to be substantially negative along the reactance axis. In the example shown, impedance measurement 612 includes a relatively low resistance value and an increasingly negative reactance value. The resistance/reactance values of impedance measurement 612, corresponding to day 16, may comprise information indicative of the wounded tissue site being in the third stage, e.g., the remodeling stage of healing. During the third stage, the impedance may remain on path 624. For example, as more epithelium grows over the wounded tissue site, the wounded tissue site may transition from being substantially conductive to being substantially capacitive (exhibiting negative reactance). In some examples, when the wounded tissue site has fully re-epithelialized, the tissue site's electrical characteristics, e.g., impedance will be similar to that of pre-wounded and/or nearby, unwounded tissue, illustrated on plot 600 as impedance 614 in the fourth, fully healed stage.

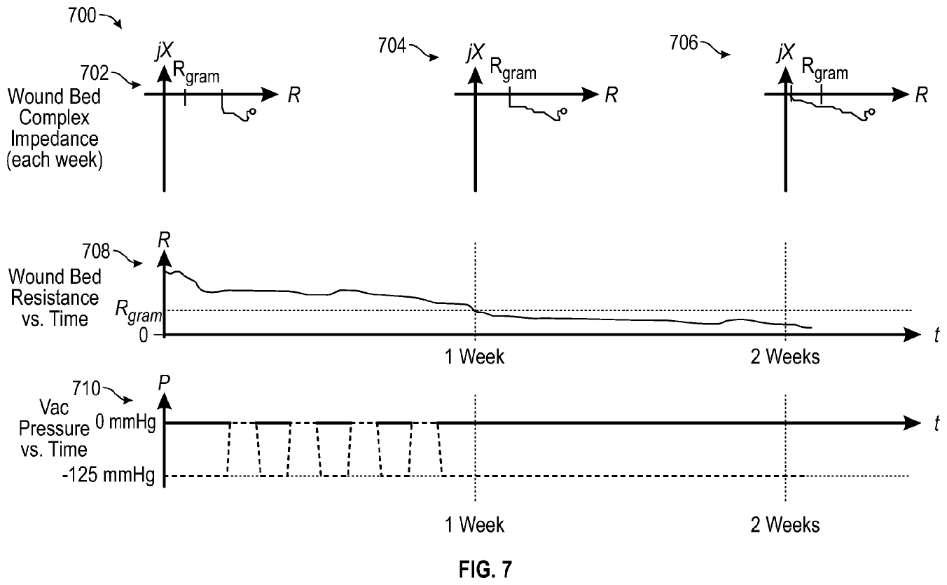

FIGS. 7-11 illustrate example scenarios of a DCL-based feedback controlled wound treatment system. In each of FIGS. 7-11, a causal model, such as causal model 110 executed by computing system 28 via DCL unit 50, may determine NPWT parameter settings 54 and/or 104 based on current causal relationships between NPWT parameter settings 54 and/or 104 and the effects of controlling the fluid at a wound site, e.g., as measured by a variety of sensors 16. In particular, the effects of controlling the fluid, e.g., healing and/or a stage of healing, may be measured by a wound bed impedance measurement system which may be integrated with NPWT dressing 20 or may be in addition to NPWT dressing 20. FIGS. 7-11 include plots illustrating impedance measurements which may indicate healing and/or a stage of healing of a wound site in conjunction with applied NPWT parameter settings.

FIG. 7 is an illustration of an example scenario 700 of wound measurements and NPWT parameter settings over a period of time, in accordance with the techniques described in this disclosure. Scenario 700 includes complex impedance measurement plots 702, 704, and 706 at different times, wound bed resistance plot 708, and the NPWT parameter setting of vacuum pressure 710.

In some examples, a DCL-based feedback controlled wound treatment system such as system 2 and/or control system 100 may use real-time wound bed impedance measurements (along with a plurality of other factors such as patient information and DCL inputs 52, wound measurements 56, and treatment targets 58) to regulate negative pressure on/off state (and other NPWT parameter settings 54 and/or 104). In some examples, control system 100 may operate within defined constraints, e.g., only being able to regulate negative pressure at two levels such as 0 mmHg or −125 mmHg, ramping pressure up or down by a constant and non-adjustable rate, and cycling negative pressure on and off cycles at a finite durations. In the example shown, the performance metric may be a decrease and/or minimization of wound bed resistance below a threshold resistance (Rgran) value indicative of the onset of granulation, and which may be related to a treatment target 58 of progressing from an inflammation stage to a proliferation stage. Control system 100 may make control decisions on the negative pressure level 710, e.g., toggling between −125 mmHg and 0 mmHg in the example shown when wound bed resistance is not dropping in value. When the wound bed does begin to granulate, as indicated by wound bed resistance dropping under the Rgran threshold, control system 100 may determine to apply a constant negative pressure of −125 mmHg.

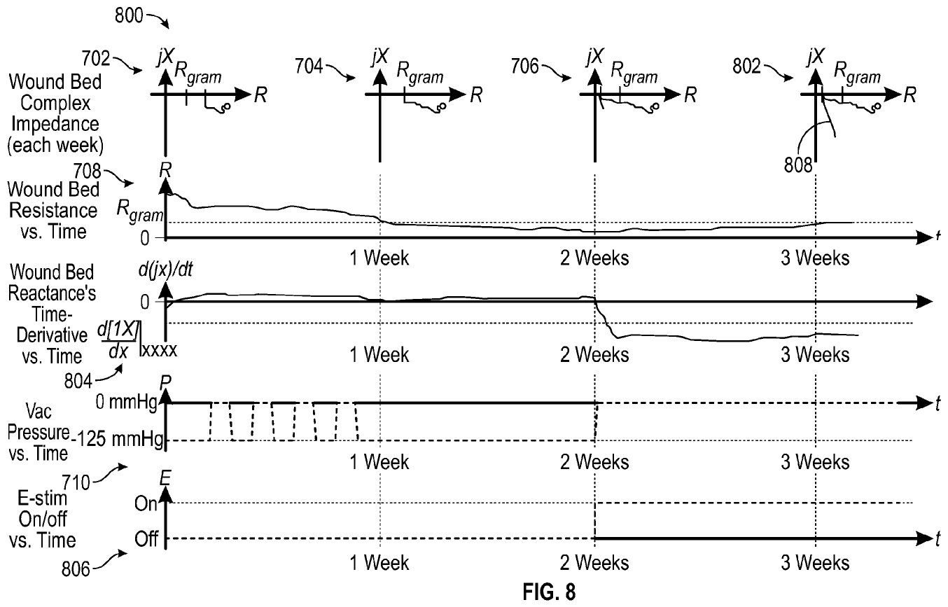

FIG. 8 is an illustration of another example scenario 800 of wound measurements and NPWT parameter settings over a period of time, in accordance with the techniques described in this disclosure. Scenario 800 includes complex impedance measurement plots 702-706 and 802 at different times, wound bed resistance plot 708, wound bed reactance time-derivative plot 804, the NPWT parameter setting of vacuum pressure 710, and electric stimulation control parameter plot 806. In the example shown, scenario 800 is continuation of scenario 700 in time.

In the example shown, if/when a wound bed has filled with an appropriate amount of granular tissue (e.g. at 2 weeks as shown), control system 100 may discontinue NPWT treatment (e.g. plot 710 returns to 0 mmHg at 2 weeks) and initiate another therapy, e.g., a therapy which may be more appropriate for re-epithelialization such as electric stimulation or oxygen delivery (e.g. plot 806 transitions from an OFF state to an ON state at 2 weeks). Control system 100 may automatically determine such a change in therapy based on current sensor data and historical data sets and pervious learnings from other patients.

In the examples shown, control system 100 may operate within the same defined constraints as described above, e.g., only being able to regulate negative pressure at two levels such as 0 mmHg or −125 mmHg, ramping pressure up or down by a constant and non-adjustable rate, and cycling negative pressure on and off cycles at finite durations. In the example shown, after granulation tissue fills the wound's void space at about 2 weeks along the time axis of the plots, epithelialization begins and the complex impedance measurements may include increasingly negative reactance values 808. The increasingly negative reactance values 808 may indicate a reduction drop in the wound bed's reactance time-derivative 804, e.g., as illustrated at 2 weeks. Control system 100 may then determine to modify control parameters by shutting off the negative pressure.

In some examples, control system 100 may include other treatment systems for increasing and/or improving re-epithelialization, such as electric stimulation or oxygen delivery. Control system 100 may determine to continue delivery of therapy by the other treatment systems if they have been used in conjunction with NPWT or to initiate delivery of these other therapies upon cessation of NPWT treatment, e.g., based on the drop in the time-derivative of the reactance 804 indicating a progression of the wound from a granulation/proliferation stage to a re-epithelialization/remodeling stage.

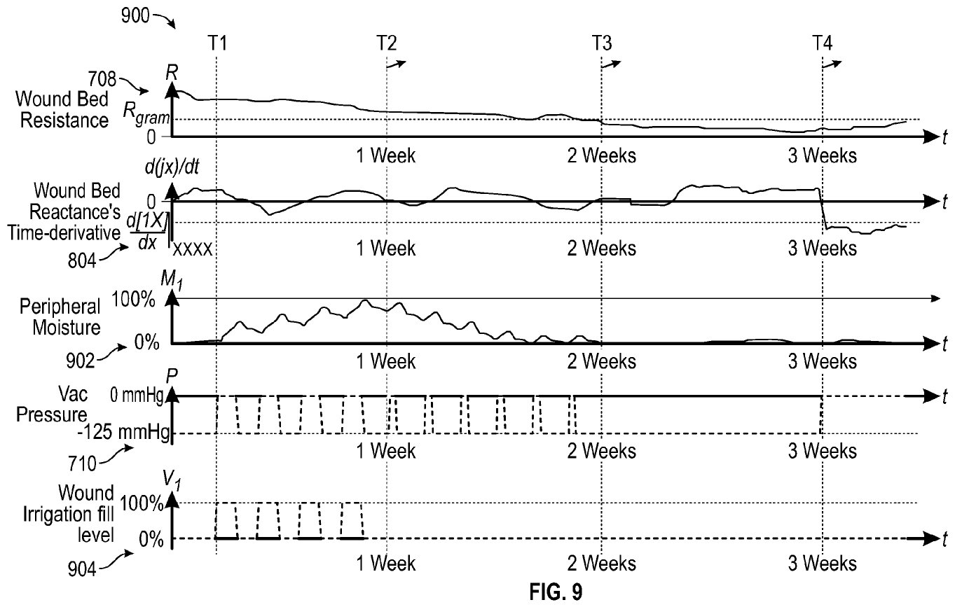

FIG. 9 is an illustration of another example scenario 900 of wound measurements and a NPWT parameter settings over a period of time, in accordance with the techniques described in this disclosure. Scenario 900 includes wound bed resistance plot 708, wound bed reactance time-derivative plot 804, the NPWT parameter setting of vacuum pressure 710, peripheral and/or peri-wound moisture versus time plot 902 and the NPWT parameter setting of wound instillation/irrigation fill level 904.

In some examples, excess moisture in the wound area may lead to peri-wound maceration, e.g., deterioration of the tissues surrounding the wound which may increase the wound size over time. A NPWT system providing wound irrigation or periodic wound instillation to enhance granulation may cause peri-wound maceration if uncontrolled and/or unaccounted for. In the example shown, control system 100 further controls NPWT parameter settings to reduce and/or eliminate peri-wound maceration.

For example, based on impedance measurements and other patient information, control system 100 may initiate a combination vacuum/instillation/dwell cycle to improve granulation at time T1. Control system 100 may flood the wound with fluid during pressure-off times, e.g., as illustrated by a 100% instillation/irrigation fill level 904 when vacuum pressure 710 is at 0 mmHg and a 0% instillation/irrigation fill level 904 when vacuum pressure 710 is at −125 mmHg.

In the example shown, control system 100 may improve granulation, e.g., as indicated by a decreasing wound bed resistance 708, between time T1 and T2. A moisture sensor 16 may measure the moisture of peri-wound tissue and the peri-wound moisture measurement 902 may indicate that the peri-wound tissue may be approaching moisture saturation from T1 to T2. Control system 100 may then modify the treatment, e.g., by shutting off the fluid irrigation/instillation. Additionally, control system 100 may change the duty cycle of vacuum pressure 710, e.g., by increasing the duration of the −125 mmHg pressure, which may help wick moisture out of the wound area.

In the example shown, peri-wound moisture 902 decreases after T2, wound bed resistance 708 decreases below a threshold value (e.g., Rgran) between times T2 and T3, e.g., indicating that the wound has progressed to the granulation stage, and the time-derivative of the wound bed's reactance 804 drops below a threshold, e.g., at time T3. Control system 100 may determine to terminate the negative pressure therapy cycling and provide a sustained/constant negative vacuum pressure 710 at time T3.

Control system 100 may determine to shut NPWT treatment off at time T4. For example, control system 100 may determine that the wound has entered a re-epithelialization/remodeling stage based on a drop of wound bed reactance time-derivative 804 at or near time T4. While described with respect to instillation as an example, it will be appreciated that various aspects of the techniques described herein with respect to FIG. 9 can also be applied to instances of wound irrigation, in which the wound is concurrently flushed with a fluid in motion and subjected to negative pressure application.

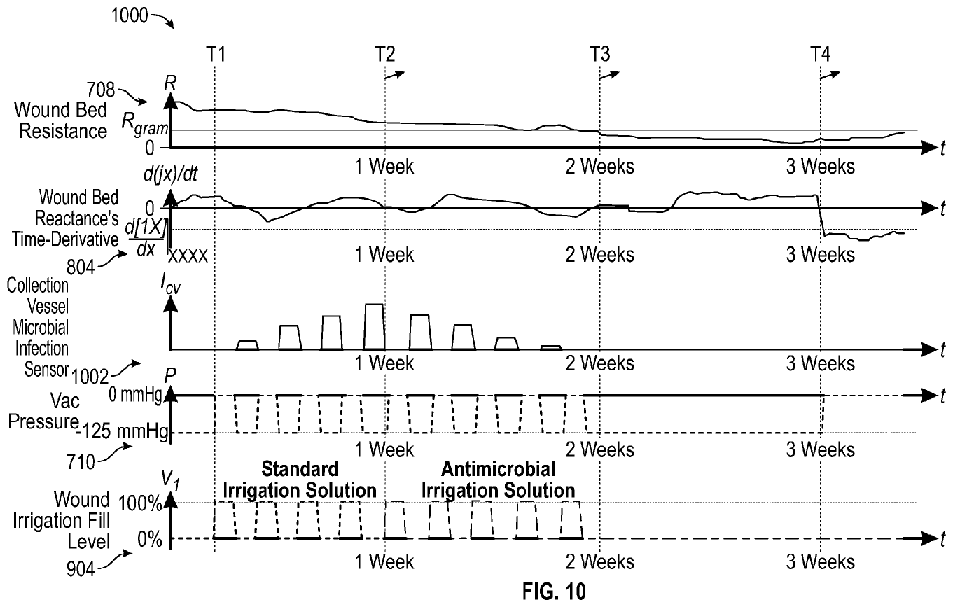

FIG. 10 is an illustration of another example scenario 1000 of wound measurements and NPWT parameter settings over a period of time, in accordance with the techniques described in this disclosure. Scenario 1000 includes wound bed resistance plot 708, wound bed reactance time-derivative plot 804, the NPWT parameter setting of vacuum pressure 710, microbial sensor plot 1002 and the NPWT parameter setting of wound instillation/irrigation fill level 904.

In some examples, during wound treatment, the wound may become infected. Control system 100 may provide rapid infection detection and treatment. For example, based on impedance measurements and other patient information, control system 100 may initiate a combination vacuum/instillation/dwell cycle to improve granulation at time T1. Control system 100 may flood the wound with a standard instillation fluid during pressure-off times, e.g., as illustrated by a 100% instillation fill level 904 when vacuum pressure 710 is at 0 mmHg and a 0% instillation fill level 904 when vacuum pressure 710 is at −125 mmHg.

Between times T1 and T2, microbial sensor data 1002 from measurement of the instillation fluid collected in the NPWT system's collection vessel may indicate an increasing probability of infection. Control system 100 may determine to infuse the standard instillation fluid with antimicrobials at time T2. Microbial sensor data 1002 from measurement of the irrigation/instillation fluid collected in the NPWT system's collection vessel may indicate a decreasing probability of infection after time T2. Control system 100 may determine to stop the antimicrobial instillation cycles at time T3.

In some examples, control system 100 may infuse the instillation fluid with any suitable agent apart from or in addition to antimicrobials, e.g., chemical curation agents, antiseptics, anti-astringents, hormones, cytokines, anti-inflammatories, immune response activators, immune response inhibitors and ionized gases. While described with respect to instillation as an example, it will be appreciated that various aspects of the techniques described herein with respect to FIG. 10 can also be applied to instances of wound irrigation, in which the wound is concurrently flushed with a fluid in motion and subjected to negative pressure application.

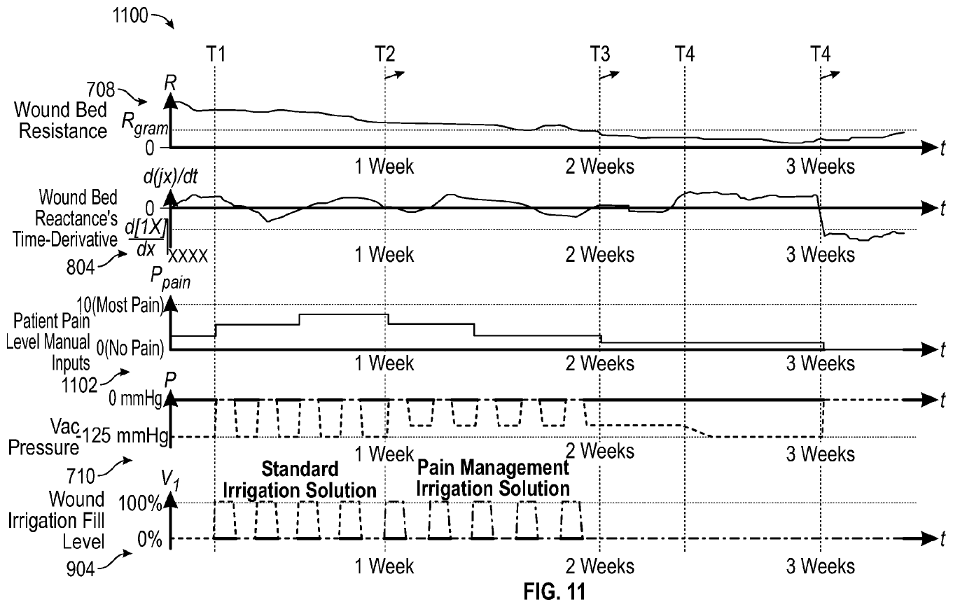

FIG. 11 is an illustration of another example scenario 1100 of wound measurements and a NPWT parameter settings over a period of time, in accordance with the techniques described in this disclosure. Scenario 1100 includes wound bed resistance plot 708, wound bed reactance time-derivative plot 804, the NPWT parameter setting of vacuum pressure 710, patient-input pain level 1102 and the NPWT parameter setting of wound instillation/irrigation fill level 904.

In some examples, control system 100 may provide customized treatment. For example, based on impedance measurements and other patient information, control system 100 may initiate a combination vacuum/irrigation cycle to improve granulation at time T1. Control system 100 may flood the wound with a standard instillation fluid during pressure-off times, e.g., as illustrated by a 100% instillation fill level 904 when vacuum pressure 710 is at 0 mmHg and a 0% instillation fill level 904 when vacuum pressure 710 is at −125 mmHg.

In some examples, the patient may input pain levels over time to control system 100. In the example shown, patient-input pain level 1102 increases between times T1 and T2. In response, control system 100 may reduce the negative pressure used during vacuum-on cycles at time T2, e.g., from −125 mmHg to −100 mmHg such that the cycle level is −100 mmHg for vacuum-on and 0 mmHg for vacuum-off as shown. In some examples, a return to ambient pressure during vacuum-off times, e.g., 0 mmHg, may cause and/or be associated with pain, and control system 100 may adjust the negative pressure used during vacuum-off cycles at time T2, e.g., from 0 mmHg to −25 mmHg (not shown), such that the cycle level is −125 mmHg (or −100 mmHg) for vacuum-on and −25 mmHg for vacuum-off. Additionally, control system 100 may replace the instillation fluid with a fluid including a pain relief medication at time T2. In some examples, control system 100 may adjust from instillation to irrigation, e.g., via providing fluid and/or fluid including a pain relief medication at a particular flow rate over a period of time.

In the example shown, patient-input pain level 1102 decreases after T2 and wound bed resistance 708 decreases below a threshold value (e.g., Rgran) between times T2 and T3, e.g., indicating that the wound has progressed to the granulation stage. Control system 100 may then determine to terminate the negative pressure therapy cycling and provide a sustained/constant negative vacuum pressure 710 at time T3, and to increase the negative vacuum pressure 710 if the patient's pain levels do not increase, e.g., at T4. While described with respect to instillation as an example, it will be appreciated that various aspects of the techniques described herein with respect to FIG. 11 can also be applied to instances of wound irrigation, in which the wound is concurrently flushed with a fluid in motion and subjected to negative pressure application.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), processing circuitry (e.g., fixed function circuitry, programmable circuitry, or any combination of fixed function circuitry and programmable circuitry), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

The following examples may illustrate one or more aspects of the disclosure:

Example 1: A method includes receiving patient information; selecting at least one negative-pressure wound therapy (NPWT) parameter setting for controlling a fluid at a wound site via a NPWT dressing based on a causal model that determines current causal relationships between a set of NPWT parameter settings and a set of effects of controlling the fluid at the wound site; and controlling the fluid at the wound site via the NPWT dressing based on the selected at least one NPWT parameter setting.

Example 2: The method of example 1 further includes receiving a measure of an effect of controlling the fluid at the wound site; and adjusting, based on the received measure of the effect of controlling the fluid at the wound site, the causal model.

Example 3: The method of example 2, wherein the at least one NPWT parameter setting is an at least one first NPWT parameter setting, the method further includes selecting, based on the adjusted causal model, at least one second NPWT parameter setting for controlling the fluid at the wound site; and controlling the fluid at the wound site via the NPWT dressing based on the selected at least one second NPWT parameter setting.

Example 4: The method of any one of examples 1-3, wherein patient information comprises at least one of user input patient information, patient biometric information, and wound measurement information, wherein wound measurement information comprises the measure of the effect of controlling the fluid at the wound site.

Example 5: The method of any of examples 1-4, wherein patient information comprises an aggregate of patient information of a plurality of patients.

Example 6: The method of any one of examples 1-5, wherein wound measurement information and the measure of the effect of controlling the fluid at the wound site comprises at least one of an impedance measurement of wounded tissue, an oxygen measurement of a wound bed, an oxygen measurement of the fluid, a carbon dioxide measurement of the wound bed, a temperature measurement, an analyte sensor measurement, and an optical measurement of the wound bed.

Example 7: The method of any one of examples 1-6, wherein the at least one first NPWT parameter setting and the at least one second NPWT parameter setting each comprise a setting of a NPWT parameter of a set of NPWT parameters, the set of NPWT parameters comprising at least one of a negative pressure level, a negative pressure cycling, a continuous negative pressure application, a fluid flow rate, a fluid volume, a fluid pressure, a fluid temperature, a fluid composition, a fluid dwell time, or a fluid purge time.

Example 8: The method of any one of examples 1-2, wherein the at least one NPWT parameter setting comprises a predetermined range of values.

Example 9: The method of any one of examples 1-8, wherein controlling the fluid at the wound site comprises providing the fluid to the wound site via the NPWT therapy dressing.

Example 10: A system includes a memory; and one or more processors in communication with the memory and configured to: receive patient information; select at least one negative-pressure wound therapy (NPWT) parameter setting based on a causal model that determines current causal relationships between a set of NPWT parameter settings and a set of effects of controlling the fluid at the wound site; and control the fluid at the wound site via the NPWT dressing based on the selected at least one NPWT parameter setting.

Example 11: The system of example 10, wherein the one or more processors are further configured to: receive a measure of an effect of controlling the fluid at the wound site; and adjust, based on the received measure of the effect of controlling the fluid at the wound site, the causal model.

Example 12: The system of example 11, wherein the at least one NPWT parameter setting is an at least one first NPWT parameter setting, wherein the one or more processors are further configured to: select based on the adjusted causal model, at least one second NPWT parameter setting for controlling the fluid at the wound site; and control the fluid at the wound site via the NPWT dressing based on the selected at least one second NPWT parameter setting.

Example 13: The system of any one of examples 10-12, wherein patient information comprises at least one of user input patient information, patient biometric information, and wound measurement information, wherein wound measurement information comprises the measure of the effect of controlling the fluid at the wound site.

Example 14: The system of any of examples 10-13, wherein patient information comprises an aggregate of patient information of a plurality of patients.

Example 15: The system of any one of examples 10-14, wherein wound measurement information and the measure of the effect of controlling the fluid at the wound site comprises at least one of an impedance measurement of wounded tissue, an oxygen measurement of a wound bed, an oxygen measurement of the fluid, a carbon dioxide measurement of the wound bed, a temperature measurement, an analyte sensor measurement, and an optical measurement of the wound bed.

Example 16: The system of any one of examples 10-15, wherein the at least one first NPWT parameter setting and the at least one second NPWT parameter setting each comprise a setting of a NPWT parameter of a set of NPWT parameters, the set of NPWT parameters comprising at least one of a negative pressure level, a negative pressure cycling, a continuous negative pressure application, a fluid flow rate, a fluid volume, a fluid pressure, a fluid temperature, a fluid composition, a fluid dwell time, or a fluid purge time.

Example 17: The system of any one of examples 10-11, wherein the at least one NPWT parameter setting comprises a predetermined range of values.

Example 18: The system of any one of examples 1-8, wherein controlling the fluid at the wound site comprises providing the fluid to the wound site via the NPWT therapy dressing.

Example 19: A computer readable medium includes instructions that when executed cause one or more processors to: receive patient information; select at least one negative-pressure wound therapy (NPWT) parameter setting based on a causal model that determines current causal relationships between a set of NPWT parameter settings and a set of effects of controlling the fluid at the wound site; and control the fluid at the wound site via the NPWT dressing based on the selected at least one first NPWT parameter setting.

Example 20: The computer readable medium of example 19, wherein the at least one NPWT parameter setting is an at least one first NPWT parameter setting, the computer readable medium further includes instructions that when executed cause one or more processors to: receive a measure of an effect of controlling the fluid at the wound site; adjust, based on the received measure of the effect of controlling the fluid at the wound site, the causal model; select based on the adjusted causal model, at least one second NPWT parameter setting for controlling the fluid at the wound site; and control the fluid at the wound site via the NPWT dressing based on the selected at least one second NPWT parameter setting.

Example 21: A system includes a means for receiving patient info; a means for selecting at least one negative-pressure wound therapy (NPWT) parameter setting for controlling a fluid at a wound site via a NPWT dressing based on a causal model that determines current causal relationships between a set of NPWT parameter settings and a set of effects of controlling the fluid at the wound site; and a means for controlling the fluid at the wound site via the NPWT dressing based on the selected at least one NPWT parameter setting.

Example 22: The system of example 21 further comprising means for performing the method of any of examples 2-9.Various examples have been described. These and other examples are within the scope of the following claims.

1. A method comprising:
receiving patient information;
selecting at least one negative-pressure wound therapy (NPWT) parameter setting for controlling a fluid at a wound site via a NPWT dressing based on a causal model that determines current causal relationships between a set of NPWT parameter settings and a set of effects of controlling the fluid at the wound site; and
controlling the fluid at the wound site via the NPWT dressing based on the selected at least one NPWT parameter setting.

2. The method of claim 1 further comprising:
receiving a measure of an effect of controlling the fluid at the wound site; and
adjusting, based on the received measure of the effect of controlling the fluid at the wound site, the causal model.

3. The method of claim 2, wherein the at least one NPWT parameter setting is an at least one first NPWT parameter setting, the method further comprising:
selecting, based on the adjusted causal model, at least one second NPWT parameter setting for controlling the fluid at the wound site; and
controlling the fluid at the wound site via the NPWT dressing based on the selected at least one second NPWT parameter setting.

4. The method of claim 1, wherein patient information comprises at least one of user input patient information, patient biometric information, and wound measurement information, wherein wound measurement information comprises the measure of the effect of controlling the fluid at the wound site.

5. The method of claim 1, wherein patient information comprises an aggregate of patient information of a plurality of patients.

6. The method of claim 1, wherein wound measurement information and the measure of the effect of controlling the fluid at the wound site comprises at least one of an impedance measurement of wounded tissue, an oxygen measurement of a wound bed, an oxygen measurement of the fluid, a carbon dioxide measurement of the wound bed, a temperature measurement, an analyte sensor measurement, and an optical measurement of the wound bed.

7. The method of claim 1, wherein the at least one first NPWT parameter setting and the at least one second NPWT parameter setting each comprise a setting of a NPWT parameter of a set of NPWT parameters, the set of NPWT parameters comprising at least one of a negative pressure level, a negative pressure cycling, a continuous negative pressure application, a fluid flow rate, a fluid volume, a fluid pressure, a fluid temperature, a fluid composition, a fluid dwell time, or a fluid purge time.

8. The method of claim 1, wherein the at least one NPWT parameter setting comprises a predetermined range of values.

9. The method of claim 1, wherein controlling the fluid at the wound site comprises providing the fluid to the wound site via the NPWT therapy dressing.

10. A system comprising:
a memory; and
one or more processors in communication with the memory and configured to:
receive patient information;
select at least one negative-pressure wound therapy (NPWT) parameter setting based on a causal model that determines current causal relationships between a set of NPWT parameter settings and a set of effects of controlling the fluid at the wound site; and
control the fluid at the wound site via the NPWT dressing based on the selected at least one NPWT parameter setting.

11. The system of claim 10, wherein the one or more processors are further configured to:
receive a measure of an effect of controlling the fluid at the wound site; and
adjust, based on the received measure of the effect of controlling the fluid at the wound site, the causal model.

12. The system of claim 11, wherein the at least one NPWT parameter setting is an at least one first NPWT parameter setting, wherein the one or more processors are further configured to:
select based on the adjusted causal model, at least one second NPWT parameter setting for controlling the fluid at the wound site; and
control the fluid at the wound site via the NPWT dressing based on the selected at least one second NPWT parameter setting.

13. The system of claim 10, wherein patient information comprises at least one of user input patient information, patient biometric information, and wound measurement information, wherein wound measurement information comprises the measure of the effect of controlling the fluid at the wound site.

14. The system of claim 10, wherein patient information comprises an aggregate of patient information of a plurality of patients.

15. The system of claim 10, wherein wound measurement information and the measure of the effect of controlling the fluid at the wound site comprises at least one of an impedance measurement of wounded tissue, an oxygen measurement of a wound bed, an oxygen measurement of the fluid, a carbon dioxide measurement of the wound bed, a temperature measurement, an analyte sensor measurement, and an optical measurement of the wound bed.

16. The system of claim 10, wherein the at least one first NPWT parameter setting and the at least one second NPWT parameter setting each comprise a setting of a NPWT parameter of a set of NPWT parameters, the set of NPWT parameters comprising at least one of a negative pressure level, a negative pressure cycling, a continuous negative pressure application, a fluid flow rate, a fluid volume, a fluid pressure, a fluid temperature, a fluid composition, a fluid dwell time, or a fluid purge time.

17. The system of claim 10, wherein the at least one NPWT parameter setting comprises a predetermined range of values.

18. The system of claim 10, wherein controlling the fluid at the wound site comprises providing the fluid to the wound site via the NPWT therapy dressing.

19. A computer readable medium comprising instructions that when executed cause one or more processors to:
receive patient information;
select at least one negative-pressure wound therapy (NPWT) parameter setting based on a causal model that determines current causal relationships between a set of NPWT parameter settings and a set of effects of controlling the fluid at the wound site; and
control the fluid at the wound site via the NPWT dressing based on the selected at least one first NPWT parameter setting.

20. The computer readable medium of claim 19, wherein the at least one NPWT parameter setting is an at least one first NPWT parameter setting, the computer readable medium further comprising instructions that when executed cause one or more processors to:
receive a measure of an effect of controlling the fluid at the wound site;
adjust, based on the received measure of the effect of controlling the fluid at the wound site, the causal model;
select based on the adjusted causal model, at least one second NPWT parameter setting for controlling the fluid at the wound site; and
control the fluid at the wound site via the NPWT dressing based on the selected at least one second NPWT parameter setting.

21-22. (canceled)
